(19) United States
(12) Patent Application Publication
(10) Pub. No.: US 2011/0251172 A1
(43) Pub. Date: Oct. 13, 2011

(54) PURINE DERIVATIVES FOR TREATMENT OF ALZHEIMER'S DISEASE

(76) Inventors: Alexey A. Rivkin, Boston, MA (US); Sean P. Ahearn, Somerville, MA (US); Stephanie M. Chichetti, Brookline, MA (US); Christohpher L. Hamblett, Boston, MA (US); Yudith Garcia, Brookline, NY (US); Michelle Martinez, Medford, MA (US); Benito Munoz, Newtonville, MA (US)

(21) Appl. No.: 13/058,232

(22) PCT Filed: Jul. 31, 2009
(86) PCT No.: PCT/US09/52323

§ 371 Date: Jun. 14, 2011

Related U.S. Application Data
(60) Provisional application No. 61/188,813, filed on Aug. 13, 2008.

(51) Int. Cl. A61K 31/52; A61P 25/28; A61K 31/551; A61K 31/55; C07D 473/16; A61K 31/5377

(52) U.S. Cl. 514/210.18; 544/277; 514/263.22; 514/252.16; 514/249; 514/210.21; 544/118; 514/234.2; 540/575; 514/218; 514/263.4; 544/230; 540/600; 514/217.06; 514/263.23; 540/594; 514/217.01

(57) ABSTRACT

The invention encompasses purine derivatives as gamma secretase modulators, useful for treating diseases associated with the deposition of beta-amyloid peptide in the brain, such as Alzheimer's disease, or of preventing or delaying the onset of dementia associated with such diseases. Pharmaceutical compositions and methods of use are included.

This invention relates to compounds for use in therapeutic treatment of the human body. In particular, it provides purine derivatives useful for treating diseases associated with the deposition of β-amyloid peptide in the brain, such as Alzheimer's disease, or of preventing or delaying the onset of dementia associated with such diseases.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. Variability in the site of the proteolysis mediated by γ-secretase results in Aβ of varying chain length, e.g. Aβ(1-38), Aβ(1-40) and Aβ(1-42). N-terminal truncations such as Aβ(4-42) are also found in the brain, possibly as a result of variability in the site of proteolysis mediated by β-secretase. For the sake of convenience, expressions such as “Aβ(1-40)” and “Aβ(1-42)” as used herein are inclusive of such N-terminal truncated variants. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD.

Other dementing conditions associated with deposition of Aβ in the brain include cerebral amyloid angiopathy, hereditary cerebral haemorrhage with amyloidosis, Dutch-type (HCHWA-D), multi-infarct dementia, dementia pugilistica and Down syndrome.

Various interventions in the plaque-forming process have been proposed as therapeutic treatments for AD (see, for example, Hardy and Selkoe, Science, 297 (2002), 353-6). One such method of treatment that has been proposed is that of blocking or attenuating the production of Aβ for example by inhibition of β- or γ-secretase. It has also been reported that inhibition of glycogen synthase kinase-3 (GSK-3), in particular inhibition of GSK-3α, can block the production of Aβ (see Phiel et al, Nature, 423 (2003), 435-9). Other proposed methods of treatment include administering a compound which blocks the aggregation of Aβ, and administering an antibody which selectively binds to Aβ.

However, recent reports (Pearson and Peers, J. Physiol., 575.1 (2006), 5-10) suggest that Aβ may exert important physiological effects independent of its role in AD, implying that blocking its production may lead to undesirable side effects. Furthermore, γ-secretase is known to act on several different substrates apart from APP (e.g. notch), and so inhibition thereof may also lead to unwanted side effects. There is therefore an interest in methods of treating AD that do not suppress completely the production of Aβ, and do not inhibit the action of γ-secretase.

One such proposed treatment involves modulation of the action of γ-secretase so as to selectively attenuate the production of Aβ(1-42). This results in preferential secretion of the shorter chain isoforms of Aβ, which are believed to have a reduced propensity for self-aggregation and plaque formation, and hence are more easily cleared from the brain, and/or are less neurotoxic. Compounds showing this effect include certain non-steroidal antiinflammatory drugs (NSAIDs) and their analogues (see WO 01/78721 and US 2002/0128319 and Weggen et al Nature, 414 (2001) 212-16; Morihara et al, J. Neurochem., 83 (2002), 1009-12; and Takahashi et al, J. Biol. Chem., 278 (2003), 18644-70). Compounds which modulate the activity of PPARα and/or PPARδ are also reported to have the effect of lowering Aβ(1-42) (WO 02/100836). NSAID derivatives capable of releasing nitric oxide have been reported to show improved anti-neuroinflammatory effects and/or to reduce intracerebral Aβ deposition in animal models (WO 02/092072; Jantzen et al, J. Neuroscience, 22 (2002), 226-54). US 2002/0015941 teaches that agents which potentiate capacitative calcium entry activity can lower Aβ(1-42).

Further classes of compounds capable of selectively attenuating Aβ(1-42) production are disclosed on WO 2005/054193, WO 2005/013985, WO 2006/008558, WO 2005/108362 and WO 2006/043064.

WO 2004/110350 discloses a variety of polycyclic compounds as suitable for modulating Aβ levels, but neither discloses nor suggests the compounds described herein.

According to the invention, there is provided a compound of formula IA or IB:

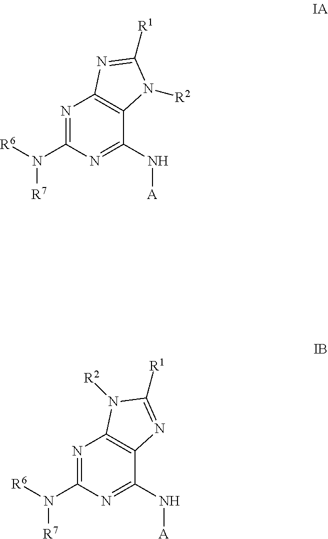

or a pharmaceutically acceptable salt or hydrate thereof; wherein:

R¹ represents H, CF₃ or C₁₋₄alkyl;

R² represents H, C₁₋₆alkyl or C₃₋₆cycloalkyl, either of which optionally bears a substituent selected from halogen, CF₃, C₁₋₄alkoxy and C₁₋₄alkoxycarbonyl;

A represents a group selected from:

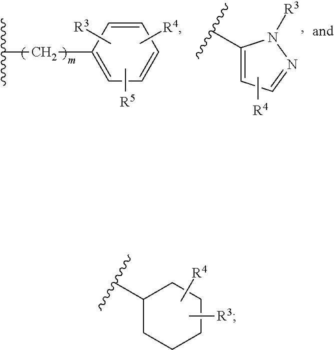

m is 0 or 1;

R³ represents C₁₋₆alkyl;

R⁴ and R⁵ are independently selected from H, halogen, C₁₋₆alkyl, C₃₋₆cycloalkyl, C₁₋₆alkoxy, C₁₋₆alkylamino and di(C₁₋₆alkyl)amino;

R⁶ represents H or C₁₋₆alkyl;

R⁷ represents —(CO)ₙ-L1-X;

n is 0 or 1;

L1 represents a divalent linking group selected from cyclopropane-1,2-diyl and C₁₋₆alkylene which optionally bears up to 2 substituents independently selected from OH, ═O, F and C₁₋₄alkyl;

X represents C₁₋₄alkoxy, C₃₋₆cycloalkylC₁₋₄alkoxy, tetrahydrofuryl, tetrahydropyranyl, Ar, ArO or ArNH;

or R⁶ and R⁷ together with the nitrogen atom which they are mutually attached complete a ring represented by:

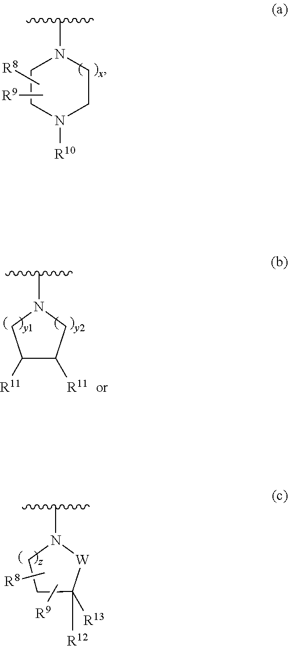

x is 1 or 2;

y1 and y2 are independently 1 or 2;

z is 0, 1 or 2;

W represents CH₂, CH₂CH₂ or CH₂CH₂O with the proviso that z=0 when W represents CH₂CH₂O;

R⁸ and R⁹ are attached to the same carbon atom or to different carbon atoms and independently represent H, halogen, CF₃, C₁₋₄alkyl or C₁₋₄alkoxy; or when attached to the same carbon atom R⁸ and R⁹ may together represent ═O; or when attached to different carbon atoms R⁸ and R⁹ may together represent a —CH₂CH₂— or —CH₂CH₂CH₂— bridge;

R¹⁰ represents a group -L2-Y;

Y represents H, Ar, OAr, NHAr, SAr, SO₂Ar, OR^{a}, N(R^{a})₂, CN, halogen, CF₃, COR^{a}, CO₂R^{a}, SO₂R^{a}, diphenylhydroxymethyl, C₃₋₆cycloalkyl, tetrahydrofuryl or tetrahydropyranyl, said C₃₋₆cycloalkyl, tetrahydrofuryl or tetrahydropyranyl optionally bearing up to 3 substituents independently selected from halogen, CF₃, C₁₋₄alkyl, oxo and C₁₋₄alkoxy;

L2 represents a bond or C₁₋₆alkylene which optionally bears up to 3 substituents selected from halogen, C₁₋₄alkyl, OH and ═O, with the proviso that L2 cannot represent a bond unless Y represents H, Ar, COR^{a}, CO₂R^{a}, SO₂R^{a} or C₃₋₆cycloalkyl;

the two R¹¹ groups together represent a fused carbocyclic or heterocyclic ring of up to 6 ring atoms in total which optionally bears up to 2 substituents independently selected from halogen, CF₃, C₁₋₄C₁₋₄alkoxy and hydroxyC₁₋₄alkyl;

R¹² represents H or a group —(Z)ₚ-L3-Y;

R¹³ represents, H, OH, Ar or C₁₋₆alkyl;

or R¹² and R¹³ together represent ═CH—Ar or ═C(Ar)₂ where the Ar groups are the same or different;

or R¹² and R¹³ together complete a spiro-linked 5-membered ring in which at least 1 of the ring atoms is N, O or S, said ring optionally being benzo-fused and said ring optionally bearing up to 2 substituents selected from oxo, Ar, CF₃, halogen, C₁₋₄alkyl, C₁₋₄alkoxy and C₁₋₄alkylcarbonyl;

Z represents O, S, SO₂, or NH;

p is 0 or 1;

L3 represents a bond or C₁₋₆alkylene which optionally bears up to 3 substituents selected from halogen, C₁₋₄alkyl, OH and ═O, with the proviso that p is 0 when L3 represents a bond;

Ar represents phenyl or 5- or 6-membered heteroaryl, any of which optionally bears up to 3 substituents selected from halogen, CN, phenyl, R^{b}, OR^{a}, N(R^{a})₂, CO₂R^{a}, CON(R^{a})₂ and SO₂R^{b};

each R^{a} independently represents H or C₁₋₄alkyl, C₃₋₆cycloalkyl, or C₃₋₆cycloalkylC₁₋₄alkyl, any of which optionally bears up to 3 fluorine substituents, or two R^{a} groups attached to the same nitrogen optionally together complete a heterocyclic ring of 4, 5 or 6 members which optionally bears up to 3 substituents independently selected from halogen, C₁₋₄alkyl, C₁₋₄alkoxy, CF₃; and oxo;

and R^{b} represents R^{a} that is other than H; or two R^{b} groups attached to adjacent ring positions may complete a fused 5- or 6-membered ring optionally bearing up to 3 substituents independently selected from halogen, CF₃, C₁₋₄alkyl, oxo and C₁₋₄alkoxy.

Where a variable occurs more than once in formula IA or IB, the identity taken by said variable at any particular occurrence is independent of the identity taken at any other occurrence.

As used herein, the expression “C₁₋ₓalkyl” where x is an integer greater than 1 refers to straight-chained and branched alkyl groups wherein the number of constituent carbon atoms is in the range 1 to x. Particular alkyl groups are methyl, ethyl, n-propyl, isopropyl and t-butyl. Derived expressions such as “C₂₋₆alkenyl”, “hydroxyC₁₋₆alkyl”, “heteroarylC₁₋₆alkyl”, “C₂₋₆alkynyl” and “C₁₋₆alkoxy” are to be construed in an analogous manner.

The expression “C₃₋₆cycloalkyl” refers to cyclic non-aromatic hydrocarbon groups containing from 3 to 6 ring carbon atoms. Examples include cyclopropyl, cyclobutyl, cyclopentenyl, cyclopentyl and cyclohexyl.

The term “heterocyclic” refers to mono- or bicyclic ring systems in which at least one ring atom is selected from N, O and S. Unless indicated otherwise, the term includes both saturated and unsaturated systems, including aromatic systems. Heterocyclic groups may be bonded via a ring carbon or a ring nitrogen, unless otherwise indicated. “Heteroaryl” refers to heterocyclic groups that are aromatic.

The term “halogen” as used herein includes fluorine, chlorine, bromine and iodine, of which fluorine and chlorine are preferred unless otherwise indicated.

For use in medicine, the compounds of formula IA or IB may be in the form of pharmaceutically acceptable salts. Other salts may, however, be useful in the preparation of these compounds or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, benzenesulphonic acid, fumaric acid, maleic acid, succinic acid, acetic acid, benzoic acid, oxalic acid, citric acid, tartaric acid, carbonic acid or phosphoric acid. Alternatively, a pharmaceutically acceptable salt may be formed by neutralisation of a carboxylic acid group with a suitable base. Examples of pharmaceutically acceptable salts thus formed include alkali metal salts such as sodium or potassium salts; ammonium salts; alkaline earth metal salts such as calcium or magnesium salts; and salts formed with suitable organic bases, such as amine salts (including pyridinium salts) and quaternary ammonium salts.

It is to be understood that all the stereoisomeric forms encompassed by formula IA and IB, both optical and geometrical, fall within the scope of the invention, singly or as mixtures in any proportion.

In the following detailed description of the invention, the variables are defined, explained and exemplified independently of each other. Hence, unless expressly indicated otherwise, any narrowed definition or particular identity disclosed for a given variable is valid in the context of every definition and identity disclosed for each of the other variables. Disclosure of any two or more overlapping sub-genera therefore discloses a further sub-genus consisting of the area of overlap between said two or more overlapping sub-genera.

In formulae IA and IB, R¹ represents H, C₁₋₄alkyl or CF₃, in particular H, Me or CF₃. Very suitably, R¹ represents H.

R² represents H, C₁₋₆alkyl or C₃₋₆cycloalkyl, either of which may by substituted with halogen, CF₃, C₁₋₄alkoxy or C₁₋₄alkoxycarbonyl. Particular identities for R² include H, methyl, ethyl, isopropyl, isobutyl, cyclopropyl, cyclobutyl, cyclopentyl, 2,2,2-trifluoroethyl and —CH₂CO₂Et.

As indicated previously, A is selected from:

(a) phenyl or benzyl which bears substituents R³, R⁴ and R⁵;

(b) 5-pyrazolyl which bears a substituent R³ on the 1-position and a substituent R⁴ elsewhere on the ring; and

(c) cyclohexyl which bears substituents R³ and R⁴.

In a particular embodiment, A represents phenyl which bears substituents R³, R⁴ and R⁵.

R³ represents C₁₋₆alkyl, such as methyl, ethyl, n-propyl, isopropyl and butyl in all its possible structural isomers. Very suitably, R³ represents methyl.

R⁴ and R⁵ independently represent H, C₁₋₆alkyl, halogen, C₃₋₆cycloalkyl, C₁₋₆alkoxy, C₁₋₆alkylamino or di(C₁₋₆alkyl)amino. Very suitably, R⁴ represents H or C₁₋₆alkyl, in particular isopropyl or t-butyl. Very suitably, R⁵ represents H or C₁₋₆alkoxy such as ethoxy.

Suitable identities of A include 2-methyl-5-t-butylphenyl, 2-methyl-5-isopropylphenyl, 2-methyl-4-ethoxy-5-isopropylphenyl, 4-t-butylbenzyl, 4-t-butylcyclohexyl and 1-methyl-3-t-butyl-1H-pyrazol-5-yl, in particular 2-methyl-5-t-butylphenyl.

In one embodiment of the invention, R⁶ represents H or C₁₋₆alkyl and R⁷ represents —(CO)ₙ-L1-X where n is 0 or 1, L1 represents a divalent linking group as defined previously, preferably C₁₋₆alkylene, and X represents C₁₋₄alkoxy, C₃₋₆cycloalkylC₁₋₄alkoxy, tetrahydrofuryl, tetrahydropyranyl, Ar, ArO or ArNH. Within this embodiment, R⁶ typically represents H or methyl, most suitably H. Examples of divalent linking groups represented by L1 include

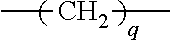

where q is 1, 2, 3 or 4, cyclopropane-1,2-diyl-CH(CH₃)CH₂—, —CH₂C(CH₃)₂CH₂—, —CH₂—C(CH₃)₂—, —CH₂CH(CH₃)— and —CH₂CH(OH)—.

Preferred identities for X include methoxy, cyclopropylmethoxy, tetrahydropyran-4-yl, Ar and ArNH, in particular Ar. Within this embodiment, Ar very suitably represents 4-methoxyphenyl, 3-bromo-4-methoxyphenyl, pyridyl (especially 3-pyridyl or 4-pyridyl), pyrazinyl, pyrimidinyl, or 5-membered nitrogen-containing heteroaryl which is optionally substituted with phenyl or C₁₋₄alkyl (especially methyl). Suitable 5-membered heteroaryl groups include imidazolyl, pyrazolyl, triazolyl, oxazolyl and thiazolyl, e.g. 1H-pyrazol-4-yl, 1-methylpyrazol-4-yl, imidazol-1-yl, 1,2,3-triazol-1-yl, 1,2,4-triazol-1-yl and 5-methyl-1,2,4-triazol-3-yl.

In a second embodiment of the invention, R⁶ and R⁷ complete a ring represented by (a):

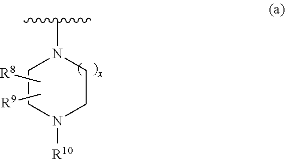

where x, R⁸, R⁹ and R¹⁰ are as defined previously. Specific examples of rings represented by (a) include:

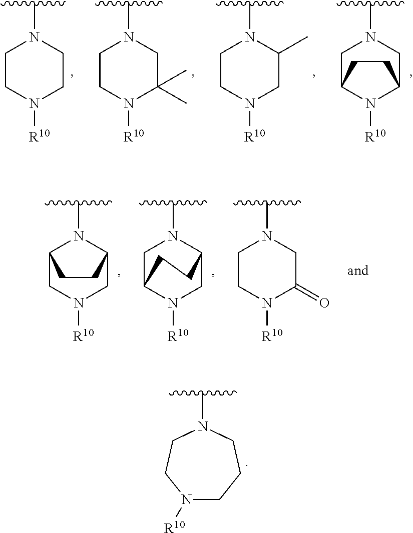

R¹⁰ represents a group -L2-Y, where L2 and Y are as defined previously. Suitable identities for L2 include a bond, CH₂, CH(CH₃), CO, CH₂CH₂, (CH₂)₃ and (CH₂)₄, but L2 cannot represent a bond unless Y represents H, Ar, COR^{a}, CO₂R^{a}, SO₂R^{a} or C₃₋₆cycloalkyl.

In a sub-embodiment, R¹⁰ represents Ar, COAr or (CH₂)ᵣ—Y¹ where r is 1, 2, 3 or 4 (in particular 1 or 2) and Y¹ represents Ar, OAr, OR^{a}, CO₂R^{a}, CON(R^{a})₂ or tetrahydropyranyl. Within this embodiment and its sub-embodiment, examples of groups represented by Ar include:

phenyl which optionally bears up to 3 substituents independently selected from halogen, CN, R^{b}, OR^{a}, CO₂R^{a}, CON(R^{a})₂ and SO₂R^{b};

pyridyl, pyrimidinyl or pyridazinyl which optionally bears a substituent selected from halogen, R^{b} and OR^{a};

and 5-membered heteroaryl optionally bearing a substituent R^{b}.

In this context, examples of suitable 5-membered heteroaryl groups include pyrazolyl, imidazolyl, triazolyl, thiazolyl, oxadiazolyl and thiadiazolyl.

Within this embodiment and its sub-embodiment, examples of groups represented by R^{a} include H, methyl, ethyl, n-propyl, isopropyl, t-butyl, cyclopropyl, cyclopropylmethyl, CF₃ and CHF₂; or two R^{a} groups attached to a single nitrogen atom complete a 4-6 membered ring, such as azetidine, pyrrolidine, 3,3-difluoropyrrolidine or 3-(trifluoromethyl)pyrrolidine. Examples of groups represented by R^{b} include methyl, ethyl, n-propyl, isopropyl, t-butyl and CF₃; or two R^{b} groups attached at adjacent ring positions can complete a fused 5- or 6-membered ring. Thus, for example, two R^{b} substituents on a phenyl ring represented by Ar may complete a methylenedioxy or ethylenedioxy group.

When Ar represents substituted phenyl, said phenyl is typically substituted in at least the 4-position, and optionally also in one or two of the 2-, 3- and 5-positions. Examples of suitable substituents include methoxy, Br, Cl, F, CN, CF₃, OCF₃, OCHF₂, SO₂Me, Me, Et, isopropyl, CO₂Et, CO₂Me, CONHMe and CONMe₂.

Particular examples of groups represented by R¹⁰ include 4-methoxyphenyl and 4-pyridylmethyl.

In a third embodiment of the invention, R⁶ and R⁷ complete a ring represented by (b):

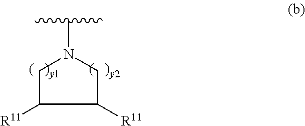

where y1, y2 and R¹¹ are as defined previously.

In a sub-embodiment y1 and y2 are the same and are either 1 or 2.

The fused ring completed by the two R¹¹ groups may be saturated or unsaturated, including aromatic, and may be carbocyclic or heterocyclic, in particular carbocyclic. Examples of fused rings completed by the two R¹¹ groups include cyclopropyl and phenyl. Specific examples of groups completed by R⁶ and R⁷ within this embodiment include 6-hydroxymethyl-3-azabicyclo[3.1.0]hex-3-yl and 7-methoxy-1,2,4,5-tetrahydro-3H-3-benzazepin-3-yl.

In a fourth embodiment of the invention, R⁶ and R⁷ complete a ring represented by (c):

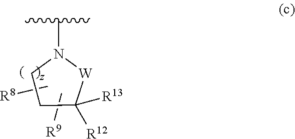

where Z, W, R⁸, R⁹, R¹² and R¹³ are as defined previously.

Rings in accordance with (c) are azetidines, pyrrolidines, piperidines, homopiperidines or morpholines bearing the substituents R⁸, R⁹, R¹² and R¹³. In a sub-embodiment, at least one of R⁸ and R⁹ is H, and in a class of this sub-embodiment R⁸ is H, F or methoxy and R⁹ is H. In a further class, R⁸ and R⁹ are both H.

R¹² represents H or a group —(Z)ₚ-L3-Y where Z, p, L3 and Y are as defined previously. When present, Z preferably represents O or S, in particular S. An alkylene chain represented by L3 preferably comprises 1-4 carbons and is unsubstituted, or bears up to two C₁₋₄alkyl substituents (e.g. methyl), or an OH substituent or an oxo (═O) substituent. When L3 is a bond, p=0 and Z is absent. Specific examples of groups represented by —(Z)ₚ-L3- include a bond, —CH₂—, —CH₂CH₂—, —CH₂CH₂CH₂—, —OCH₂—, —OCH₂CH₂—, —COCH₂—, and —CH(OH)CH₂—.

Within this embodiment and its subembodiment, particular identities for Y include Ar, OAr, SAr, SO₂Ar, CN, N(R^{a})₂, OR^{a}, CO₂R^{a}, CON(R^{a})₂, C₃₋₆cycloalkyl (such as 4-methoxycyclohexyl or 4-oxocyclohexyl) and diphenylhydroxymethyl where R^{a} and Ar are as defined previously. Preferred identities for R^{a} include H, methyl, ethyl, n-propyl, isopropyl and cyclopropyl, or two R^{a} groups attached to the same nitrogen complete a ring, in particular pyrrolidine which optionally has an oxo-substituent in the 2-position. Particular identities for Ar include:

phenyl which optionally bears up to 3 substituents selected from halogen, CN, R^{b}, OR^{a}, CO₂R^{a}, CON(R^{a})₂ and SO₂R^{b};

pyridyl or pyrimidinyl which optionally bears up to 2 substituents selected from halogen, R^{b} and OR^{a};

and 5-membered heteroaryl optionally bearing up to 2 R^{b} substituents.

Preferred 5-membered heteroaryl groups include imidazole, pyrazole, triazole (especially 1,2,3-trazole) and oxadiazole, optionally substituted with up to 2 C₁₋₄ alkyl groups.

In a particular class of this embodiment, Ar is selected from phenyl, 4-methoxyphenyl, 4-chlorophenyl, 4-trifluoromethylphenyl, 4-methanesulfonylphenyl, pyridyl which is optionally substituted with F, methyl or methoxy, or pyrimidinyl which is optionally substituted with methyl, ethyl or methoxy, or imidazole, pyrazole, triazole or oxadiazole which are optionally substituted with up to 2 independent methyl or ethyl groups.

R¹³ represents H, OH, Ar, or C₁₋₆alkyl (such as methyl). When R¹³ is Ar, R¹² is preferably Ar or SO₂Ar. In a particular class, R¹³ is H.

Alternatively, R¹² and R¹³ complete a group represented by ═CHAr or ═C(Ar)₂ where the Ar groups are the same or different. In this context, specific identities for Ar include phenyl and 4-pyridyl.

In a further alternative, R¹² and R¹³ complete a spiro-linked 5-membered ring in which at least one of the ring atoms is N, O or S (in particular N or O), said ring optionally being benzo-fused and optionally bearing up to 2 substituents selected from oxo, Ar, CF₃, halogen, C₁₋₄alkyl, C₁₋₄alkoxy and C₁₋₄alkylcarbonyl. Examples of such spiro-linked rings include:

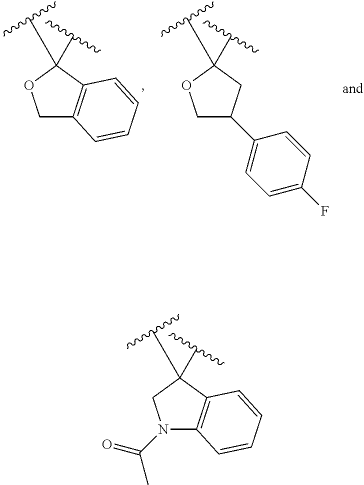

Specific examples of compounds in accordance with the invention are provided in the Examples section.

Compounds of formula IA and IB may be prepared by reaction of purine derivatives (1) and (2), respectively, with R⁶R⁷NH

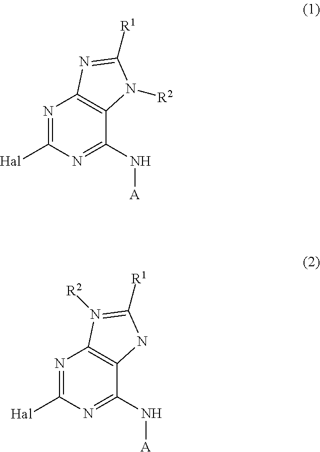

where Hal represents Cl, Br or I and R¹, R², A, R⁶ and R⁷ have the same meanings as before. The reaction takes place in an alkanol solvent (e.g. isopropanol) with microwave heating (e.g. at about 160° C.) in the presence of a tertiary amine (e.g. diisopropylethylamine). Alternatively, the reaction may be carried out under Buchwald conditions, i.e. with heating in a solvent such as toluene or dioxan in the presence of base (such as sodium carbonate) and Pd(0) and phosphine catalysts. Suitable catalysts include tris(dibenzylideneacetone)dipalladium(0) and 4,5-bis(diphenylphosphino)-9,9-dimethylxanthene.

Compounds (1) and (2) may be prepared similarly by treatment of dihalides (3) and (4) with A-NH₂:

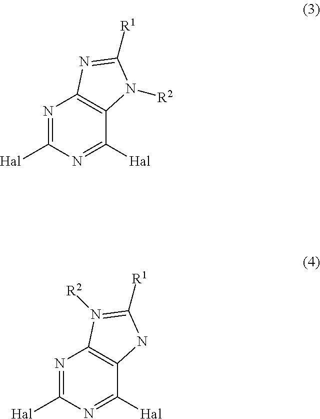

where Hal, R¹, R² and A have the same meanings as before. The reaction may be carried out by heating (e.g. in the range 80-120° C.) in the presence of a tertiary amine (e.g. triethylamine or diisopropylethylamine), either neat or in an alkanol solvent such as 2-propanol.

Alternatively, dihalide (3) or (4) may be reacted with R⁶R⁷NH and then with Ar—NH₂.

It will be apparent to those skilled in the art that the conventional techniques of organic synthesis may be used to convert individual compounds in accordance with formula IA and IB into other compounds also in accordance with formula IA or IB. Such techniques include ester or amide formation or hydrolysis, oxidation, reduction, alkylation and carbon-carbon bond formation via coupling or condensation. Such techniques may similarly be applied to the synthetic precursors of compounds of formula I.

Where they are not themselves commercially available, the starting materials for the synthetic schemes described above are available by straightforward chemical modifications of commercially available materials.

Certain compounds according to the invention may exist as optical isomers due to the presence of one or more chiral centres or because of the overall asymmetry of the molecule. Such compounds may be prepared in racemic form, or individual enantiomers may be prepared either by enantiospecific synthesis or by resolution. The novel compounds may, for example, be resolved into their component enantiomers by standard techniques such as preparative HPLC, or the formation of diastereomeric pairs by salt formation with an optically active acid, such as di-p-toluoyl-D-tartaric acid and/or di-p-toluoyl-L-tartaric acid, followed by fractional crystallisation and regeneration of the free base. The novel compounds may also be resolved by formation of diastereomeric esters or amides, followed by chromatographic separation and removal of the chiral auxiliary. Alternatively, racemic intermediates in the preparation of compounds of formula I may be resolved by the aforementioned techniques, and the desired enantiomer used in subsequent steps.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W, McOmie, Plenum Press, 1973; and T. W. Greene & P. G. M. Wuts, Protective Groups in Organic Synthesis, John Wiley & Sons, 3^{rd} ed., 1999. The protecting groups may be removed at a convenient subsequent stage using methods known from the art.

The compounds of the invention have the useful property of modifying the action of γ-secretase on amyloid precursor protein so as to selectively reduce the formation of the 1-42 isoform of Aβ, and hence find use in the development of treatments for diseases mediated by Aβ(1-42), in particular diseases involving deposition of β-amyloid in the brain.

According to a further aspect of the invention there is provided the use of a compound according to formula IA or IB as defined above, or a pharmaceutically acceptable salt or hydrate thereof, for the manufacture of a medicament for treatment or prevention of a disease associated with the deposition of β-amyloid in the brain.

The disease associated with deposition of Aβ in the brain is typically Alzheimer's disease (AD), cerebral amyloid angiopathy, HCHWA-D, multi-infarct dementia, dementia pugilistica or Down syndrome, preferably AD.

In a further aspect, the invention provides the use of a compound of Formula IA or IB as defined above, or a pharmaceutically acceptable salt or hydrate thereof, in the manufacture of a medicament for treating, preventing or delaying the onset of dementia associated with Alzheimer's disease, cerebral amyloid angiopathy, HCHWA-D, multi-infarct dementia, dementia pugilistica or Down syndrome.

The invention also provides a method of treating or preventing a disease associated with deposition of Aβ in the brain comprising administering to a patient in need thereof a therapeutically effective amount of a compound of Formula IA or IB as defined above or a pharmaceutically acceptable salt or hydrate thereof.

In a further aspect, the invention provides a method of treating, preventing or delaying the onset of dementia associated with Alzheimer's disease, cerebral amyloid angiopathy, HCHWA-D, multi-infarct dementia, dementia pugilistica or Down syndrome comprising administering to a patient in need thereof a therapeutically effective amount of a compound of Formula IA or IB as defined above or a pharmaceutically acceptable salt or hydrate thereof.

The compounds of Formula IA or IB modulate the action of γ-secretase so as to selectively attenuate production of the (1-42) isoform of Aβ without significantly lowering production of the shorter chain isoforms such as Aβ(1-40). This results in secretion of Aβ which has less tendency to self-aggregate and form insoluble deposits, is more easily cleared from the brain, and/or is less neurotoxic. Therefore, a further aspect of the invention provides a method for retarding, arresting or preventing the accumulation of Aβ in the brain comprising administering to a subject in need thereof a therapeutically effective amount of a compound of Formula IA or IB as defined above or a pharmaceutically acceptable salt thereof.

Because the compounds of Formula IA or IB modulate the activity of γ-secretase, as opposed to suppressing said activity, it is believed that the therapeutic benefits described above will be obtained with a reduced risk of side effects, e.g. those that might arise from a disruption of other signalling pathways (e.g. Notch) which are controlled by γ-secretase.

In one embodiment of the invention, the compound of Formula IA or IB is administered to a patient suffering from AD, cerebral amyloid angiopathy, HCHWA-D, multi-infarct dementia, dementia pugilistica or Down syndrome, preferably AD.

In an alternative embodiment of the invention, the compound of Formula IA or IB is administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neural., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neural., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the compound of Formula IA or IB is advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, the compound of Formula IA or IB is administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42),

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

The compounds of Formula IA or IB are typically used in the form of pharmaceutical compositions comprising one or more compounds of Formula IA or IB and a pharmaceutically acceptable carrier. Accordingly, in a further aspect the invention provides a pharmaceutical composition comprising a compound of Formula IA or IB as defined above, or a pharmaceutically acceptable salt thereof, and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyethylene glycol), polyvinylpyrrolidone) or gelatin.

For treating or preventing Alzheimer's disease, a suitable dosage level is about 0.01 to 250 mg/kg per day, preferably about 0.01 to 100 mg/kg per day, and more preferably about 0.05 to 50 mg/kg of body weight per day, of the active compound. The compounds may be administered on a regimen of 1 to 4 times per day. In some cases, however, a dosage outside these limits may be used.

The compounds of Formula IA or IB optionally may be administered in combination with one or more additional compounds known to be useful in the treatment or prevention of AD or the symptoms thereof. Such additional compounds thus include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878). Such additional compounds also include cholesterol-lowering drugs such as the statins, e.g. simvastatin. Such additional compounds similarly include compounds known to modify the production or processing of Aβ in the brain (“amyloid modifiers”), such as compounds which inhibit the secretion of Aβ (including γ-secretase inhibitors, β-secretase inhibitors, and GSK-3α inhibitors), compounds which inhibit the aggregation of Aβ, and antibodies which selectively bind to Aβ. Such additional compounds also include growth hormone secretagogues, as disclosed in WO 2004/110443.

In this embodiment of the invention, the amyloid modifier may be a compound which inhibits the secretion of Aβ, for example an inhibitor of γ-secretase (such as those disclosed in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, WO 2004/089911, WO 02/081435, WO 02/081433, WO 03/018543, WO 2004/031137, WO 2004/031139, WO 2004/031138, WO 2004/101538, WO 2004/101539 and WO 02/47671), or a β-secretase inhibitor (such as those disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621, and also including GSK-3 inhibitors, particularly GSK-3α inhibitors, such as lithium, as disclosed in Phiel et al, Nature, 423 (2003), 435-9.

Alternatively, the amyloid modifier may be a compound which inhibits the aggregation of Aβ or otherwise attenuates is neurotoxicicity. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 (in particular 3-aminopropane-1-sulfonic acid, also known as tramiprosate or Alzhemed™); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191. Further examples include phytic acid derivatives as disclosed in U.S. Pat. No. 4,847,082 and inositol derivatives as taught in US 2004/0204387.

Alternatively, the amyloid modifier may be an antibody which binds selectively to Aβ. Said antibody may be polyclonal or monoclonal, but is preferably monoclonal, and is preferably human or humanized. Preferably, the antibody is capable of sequestering soluble Aβ from biological fluids, as described in WO 03/016466, WO 03/016467, WO 03/015691 and WO 01/62801. Suitable antibodies include humanized antibody 266 (described in WO 01/62801) and the modified version thereof described in WO 03/016466.

As used herein, the expression “in combination with” requires that therapeutically effective amounts of both the compound of Formula IA or IB and the additional compound are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional compound is an antibody, it will typically be administered parenterally and separately from the compound of Formula IA or IB.

### EXAMPLES

The ability of the compounds of Formula Ito selectively inhibit production of Aβ(1-42) may be determined using the following assay:

### Cell-Based γ-Secretase Assay

Human SH-SY5Y neuroblastoma cells overexpressing the direct γ-secretase substrate SPA4CT were induced with sodium butyrate (10 mM) for 4 hours prior to plating. Cells were plated at 35,000 cells/well/100 μl in 96-well plates in phenol red-free MEM/10% FBS, 50 mM HEPES, 1% Glutamine and incubated for 2 hrs at 37° C., 5% CO₂.

Compounds for testing were diluted into Me₂SO to give a ten point dose-response curve. Typically 10 μl of these diluted compounds in Me₂SO were further diluted into 182 μl dilution buffer (phenol red-free MEM/10% FBS, 50 mM HEPES, 1% Glutamine) and 10 μl of each dilution was added to the cells in 96-well plates (yielding a final Me₂SO concentration of 0.5%). Appropriate vehicle and inhibitor controls were used to determine the window of the assay.

After incubation overnight at 37° C., 5% CO₂, 25 μl and 50 μl media were transferred into a standard Meso avidin-coated 96-well plate for detection of Aβ(40) and Aβ(42) peptides, respectively. 25 μl Meso Assay buffer (PBS, 2% BSA, 0.2% Tween-20) was added to the Aβ(40) wells followed by the addition of 25 μl of the respective antibody premixes to the wells:

Aβ(40) premix: 1 μg/ml ruthenylated G2-10 antibody, 4 μg/ml

biotinylated 4G8 antibody diluted in Origen buffer

Aβ(42) premix: 1 μg/ml ruthenylated G2-11 antibody, 4 μg/ml

biotinylated 4G8 antibody diluted in Origen buffer

(Biotinylated 4G8 antibody supplied by Signet Pathology Ltd; G2-10 and G2-11 antibodies supplied by Chemicon)

After overnight incubation of the assay plates on a shaker at 4° C., the Meso Scale Sector 6000 Imager was calibrated according to the manufacturer's instructions. After washing the plates 3 times with 150 μl of PBS per well, 150 μl Meso Scale Discovery read buffer was added to each well and the plates were read on the Sector 6000 Imager according to the manufacturer's instructions.

Cell viability was measured in the corresponding cells after removal of the media for the Aβ assays by a colorimetric cell proliferation assay (CellTiter 96™ AQ assay, Promega) utilizing the bioreduction of MTS (Owen's reagent) to formazan according to the manufacturer's instructions. Briefly, 5 μl of 10×MTS/PES was added to the remaining 50 μl of media before returning to the incubator. The optical density was read at 495 nm after ˜4 hours.

LD₅₀ and IC₅₀ values for inhibition of Aβ(40) and Aβ(42) were calculated by nonlinear regression fit analysis using the appropriate software (eg. Excel fit). The total signal and the background were defined by the corresponding Me₂SO and inhibitor controls.

The compounds listed in the following examples all gave IC₅₀ values for Aβ(1-42) inhibition of less than 10 μM and in most cases less than 1.0 μM. Furthermore, said values were at least 2-fold lower than the corresponding IC₅₀ values for Aβ(1-40) inhibition, typically at least 5-fold lower, and in the preferred cases up to 50-fold lower.

### Assay for In Vivo Efficacy

APP-YAC transgenic mice (20-30 g; 2-6 months old) and Sprague Dawley rats (200-250 g; 8-10 weeks old) were kept on 12-hr light/dark cycle with unrestricted access to food and water. Mice and rats were fasted overnight and were then dosed orally at 10 ml/kg with test compound formulated in either imwitor:Tween-80 (50:50) or 10% Tween-80, respectively. For compound screening studies, test compounds were administered at a single dose (20 or 100 mg/kg) and blood was taken serially at 1 and 4 hrs via tail bleed from mice and terminally at 7 hrs for mice and rats via cardiac puncture. In dose response studies, compounds were given at 0.1, 3, 10, 30, and 100 mg/kg and blood was taken terminally at 7 hrs from mice and rats via cardiac puncture. Following euthanasia by CO₂, forebrain tissue was harvested from animals and stored at −80 degrees. For PD analysis of brain Aβ levels, soluble Aβ was extracted from hemi-forebrains by homogenization in 10 volumes of 0.2% DEA in 50 mM NaCl followed by ultracentrifugation. Levels of Aβ 42/40 were analyzed using Meso Scale technology (electrochemiluminesence) with biotinylated 4G8 capture antibody and ruthenium labeled 12F4 or G210 detection antibodies for Aβ 42 and Aβ 40, respectively. For PK analysis, blood and brain samples were processed using a protein precipitation procedure with the remaining filtrate being analyzed via LC/MS/MS to determine drug exposure levels, brain penetration, and ED50/EC50, where appropriate.

### Example 1

### N-(5-tert-butyl-2-methylphenyl)-2-[4-(4-methoxyphenyl)-3,3-dimethylpiperazin-1-yl]-7-methyl-7H-purin-6-amine

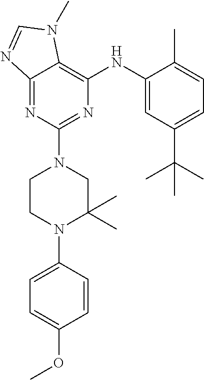

### Step 1: N-(5-tert-butyl-2-methylphenyl)-2-chloro-7-methyl-7H-purin-6-amine

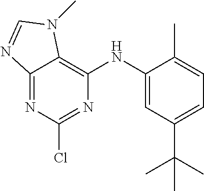

A solution of 2,6-dichloro-7-methyl-7H-purine (10.0 g, 49.3 mmol), 2-methyl-5-t-butylaniline (12.06 g, 73.9 mmol) and diisopropylethylamine (100 mL) in 2-propanol (100 mL) was heated at 120° C. for 17 h in an oil bath. The mixture was cooled to room temperature and then cooled further in the refrigerator for 2 hrs. The precipitate was filtered off and washed with cooled 2-propanol (25 mL). The precipitate was collected and dried under vacuum to afford the product (9.12 g, 54.5%) as a solid. LCMS and NMR showed that the precipitate was the desired product. LC-ESMS observed [M+H]+ 330.1 (calcd 330.1).

### Step 2: N-(5-tert-butyl-2-methylphenyl)-2-[4-(4-methoxyphenyl)-3,3-dimethylpiperazin-1-yl]-7-methyl-7H-purin-6-amine

A solution of the product of Step 1 (1.0 g, 3.03 mmol), 1-(4-methoxyphenyl)-2,2-dimethylpiperazine.2HCl (1.51 g, 5.15 mmol) and diisopropylethylamine (5.0 mL) was irradiated in 2-propanol (5 mL), in a microwave oven, at 180° C. for 2.5 hrs. The mixture was cooled and the solvent was evaporated under reduced pressure. The residue was purified by column chromatography on silica gel (Biotage system, MeOH/dichloromethane, 0%-100%) to give the product (1.14 g, 73%) as a solid. ¹H-NMR (600 MHz, CDCl₃) δ=0.984 (6H, s), 1.29 (9H, s), 2.29 (3H, s), 3.09 (2H, t, J=5.2 Hz), 3.71 (2H, s), 3.76 (3H, s), 3.81 (3H, s), 3.94 (2H, t, J=5.0 Hz), 6.32 (1H, s), 6.78 (2H, d, J=7.8 Hz), 7.04 (2H, d, J=7.8 Hz), 7.08 (1H, dd, J=7.9, 2.0 Hz), 7.15 (1H, d, J=7.9 Hz), 7.62 (1H, s), 7.82 (1H, s).

LC-ESMS observed [M+H]+ 514.3 (calcd 514.3).

### Example 2

### N-(5-tert-butyl-2-methylphenyl)-7-methyl-2-{4-[2-(4-methyl-1H-imidazol-1-yl)ethyl]piperidin-1-yl}-7H-purin-6-amine

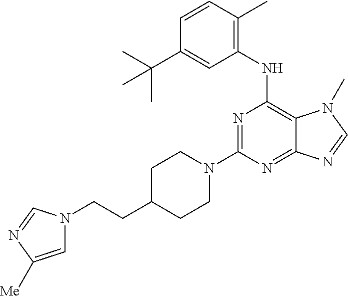

### Step 1

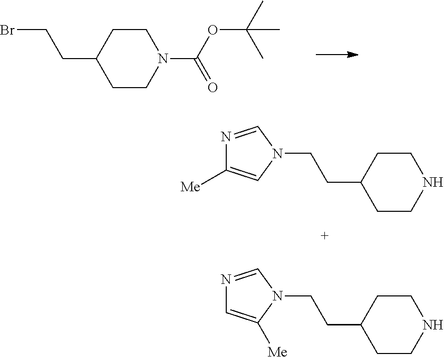

To a solution of 4-methyl-1H-imidazole (100 mg, 1.22 mmol) and sodium hydride (60 mg, 1.49 mmol) in DMF (6 mL) was added tert-butyl-4-(2-bromoethyl)piperidine-1-carboxylate (427 mg, 1.46 mmol) and potassium iodide (40 mg, 0.24 mmol). The reaction mixture was heated at 80° C. for 2 h and then cooled to RT and stirred overnight. The reaction mixture was diluted with CH₂Cl₂ (10 mL) and washed with H₂O (1×10 mL). The aqueous layer was further extracted with CH₂Cl₂ (1×10 mL) and the combined organics dried over Na₂SO₄, filtered, and concentrated. The crude material was purified by flash chromatography (2-10% MeOH/CH₂Cl₂) to give an inseparable mixture of imidazole regioisomers confirmed by MS (ESI+): cal'd [M+H]⁺ 294.2, obs. 294.2. The mixture was treated with TFA (1 mL) in CH₂Cl₂ (2 mL) and stirred overnight at RT. The reaction mixture was then concentrated and purified by flash chromatography (5-15% MeOH/CH₂Cl₂) to give 236 mg (99%) of a 2:1 mixture of isomers favoring the external methyl imidazole confirmed by MS (ESI+): cal'd [M+H]⁺ 194.2, obs. 194.1.

### Step 2

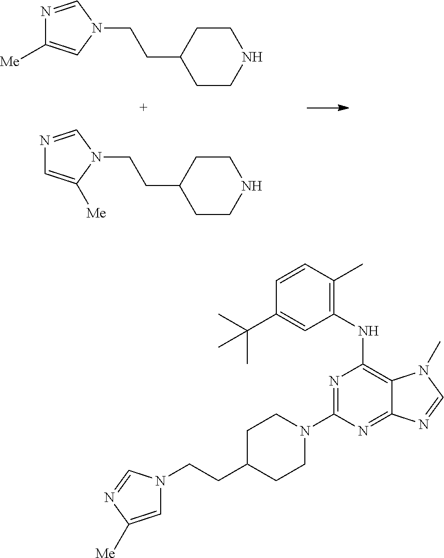

A mixture of the imidazole regioisomers (88 mg, 0.46 mmol), Hünig's base (1.5 mL), and the chloro methyl purine of Example 1 Step 1 (100 mg, 0.303 mmol) in 2-propanol (1.5 mL) was heated for 2 h at 190° C. in the microwave. The reaction mixture was then concentrated and purified by flash chromatography (2-10% MeOH/CH₂Cl₂, □=210 nM) to give 87 mg (59%) of a 2:1 mixture of amino methyl purine imidazole regioisomers, which were separated by HPLC (Chiralcel OJ, 30% EtOH/heptane, flow rate=0.75 mL/min, □=254 nM, t_{R}=8.76 min. (major); t_{R}=12.76 min. (minor)) and confirmed by MS (ESI+): cal'd [M+H]⁺ 487.3, obs. 487.2 and ¹HNMR (CDCl₃) □7.83 (d, 1H, J=1.1 Hz), 7.62 (s, 1H), 7.53 (s, 1H), 7.14 (d, 1H, J=8.0 Hz), 7.08 (dd, 1H, J₁=7.9 Hz, J₂=1.5 Hz), 6.62 (s, 1H), 6.34 (s, 1H), 4.78 (d, 2H, J=13.1), 3.91 (t, 2H, J=7.4), 3.81 (s, 3H), 2.76 (t, 2H, J=12.0 Hz), 2.28 (s, 3H), 2.22 (s, 3H), 1.70 (q, 2H, J=7.5 Hz), 1.67 (br d, 2H, J=12.7 Hz), 1.56-1.37 (m, 1H), 1.31-1.11 (m, 13H).

### Example 3

### N-(5-tert-butyl-2-methylphenyl)-7-methyl-2-{4-[(6-methylpyrimidin-4-yl)oxy]piperidin-1-yl}-7H-purin-6-amine

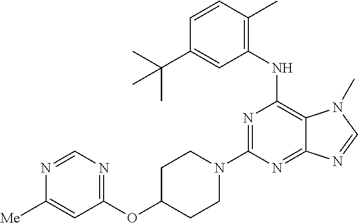

### Step 1: 4-methyl-6-(piperidin-4-yloxy)pyrimidine

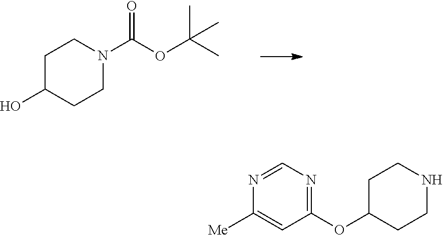

To a solution of tert-butyl 4-hydroxypiperidine-1-carboxylate (600 mg, 2.98 mmol) in DMF (6 mL) was added sodium hydride (131 mg, 3.28 mmol) followed by 4-chloro-6-methylpyrimidine (383 mg, 2.98 mmol). The reaction mixture was heated at 80° C. for 2 h and then diluted with CH₂Cl₂ (10 mL) and washed with H₂O (1×10 mL), brine (1×10 mL) and then dried over Na₂SO₄, filtered, and concentrated. The crude material was purified by flash chromatography (2-10% MeOH/CH₂Cl₂) to give tert-butyl 4-[(6-methylpyrimidin-4-yl)oxy]piperidine-1-carboxylate confirmed by MS (ESI+): cal'd [M+H]⁺ 294.2, obs. 294.2, which was treated with 4M HCl as a solution in dioxane (2 mL) and then concentrated to afford 4-methyl-6-(piperidin-4-yloxy)pyrimidine confirmed by MS (ESI+): cal'd [M+H]⁺ 194.1, obs. 194.2.

### Step 2

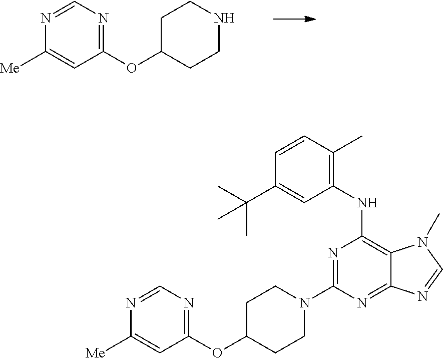

A mixture of 4-methyl-6-(piperidin-4-yloxy)pyrimidine (119 mg, 0.39 mmol), Hünig's base (1.5 mL), and the chloro methyl purine (100 mg, 0.30 mmol) in 2-propanol (1.5 mL) was heated for 2 hr at 190° C. The mixture was concentrated and purified by flash chromatography (2-15% MeOH/CH₂Cl₂) to give 67 mg (45%) of the desired amino methyl purine confirmed by MS (ESI+): cal'd [M+H]⁺ 487.3, obs. 487.3 and ¹H NMR (CD₃OD) □ 8.53 (d, 1H, J=0.8 Hz), 7.88 (s, 1H), 7.51 (s, 1H), 7.16 (ABq, J=7.2 Hz), 6.69 (s, 1H), 5.29 (tt, 1H, J₁=8.3 Hz, J₂=4.1 Hz), 4.11 (dt, 2H, J₁=13.9 Hz, J₂=4.9 Hz), 4.01 (s, 3H), 3.35 (ddd, 2H, J₁=13.3 Hz, J₂=9.2 Hz, J₃=3.4 Hz), 2.40 (s, 3H), 2.24 (s, 3H), 2.00-1.88 (m, 2H), 1.62 (dddd, 2H, J₁=12.8 Hz, J₂=8.6 Hz, J₃=8.6 Hz, J₄=3.9 Hz), 1.28 (s, 9H).

### Examples 4-231

The compounds in the following table were prepared by the same route, using the appropriate aniline and dichloropurine derivatives in the procedure of Step 1 of Example 1, and the appropriate amine derivatives in the procedure of Step 2.

| | | | |
|---|---|---|---|
| 4 | 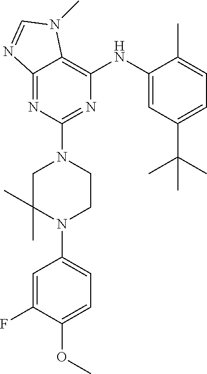 | N-(5-tert-butyl-2- methylphenyl)-2-[4-(3-fluoro- 4-methoxyphenyl)-3,3- dimethylpiperazin-1-yl]-7- methyl-7H-purin-6-amine | Calc'd 532.3, found 532.1 |
| 5 | 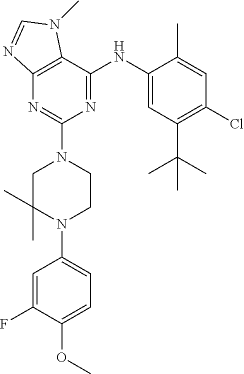 | N-(5-tert-butyl-4-chloro-2- methylphenyl)-2-[4-(3-fluoro- 4-methoxyphenyl)-3,3- dimethylpiperzin-1-yl]-7- methyl-7H-purin-6-amine | Calc'd 566.3, found 566.1 |
| 6 | 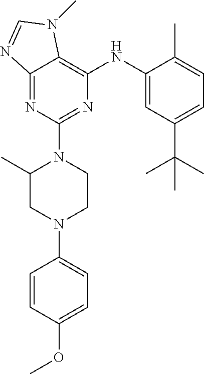 | N-(5-tert-butyl-2- methylphenyl)-2-[4-(4- methoxyphenyl)-2- methylpiperazin-1-yl]-7- methyl-7H-purin-6-amine | Calc'd 500.3, found 500.1 |
| 7 | 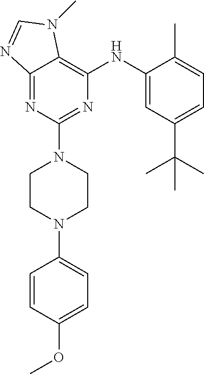 | N-(5-tert-butyl-2- methylphenyl)-2-[4-(4- methoxyphenyl)piperazin-1- yl]-7-methyl-7H-purin- 6-amine | Calc'd 486.3, found 486.1 |
| 8 | 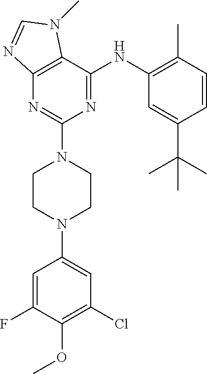 | N-(5-tert-butyl-2- methylphenyl)-2-[4-(3-chloro- 5-fluoro-4-methoxyphenyl) piperazin-1-yl]-7-methyl-7H- purin-6- amine | Calc'd 538.2, found 538.0 |
| 9 | 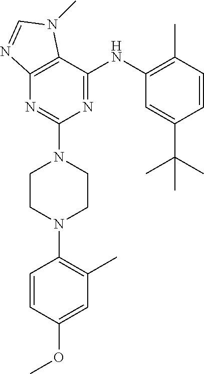 | N-(5-tert-butyl-2- methylphenyl)-2-[4-(4- methoxy-2-methylphenyl) piperazin-1-yl]-7-methyl-7H- purin-6-amine | Calc'd 500.3, found 500.1 |
| 10 | 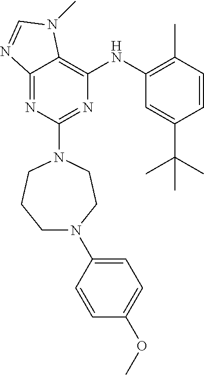 | N-(5-tert-butyl-2- methylphenyl)-2-[4-(4- methoxyphenyl)-1,4-diazepan- 1-yl]-7-methyl-7H-purin-6- amine | Calc'd 500.3, found 500.1 |
| 11 | 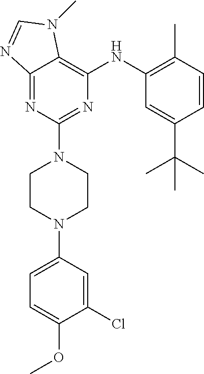 | N-(5-tert-butyl-2- methylphenyl)-2-[4-(3-chloro- 4-methoxyphenyl)piperazin-1- yl]-7-methyl-7H-purin-6- amine | Calc'd 520.3, found 520.1 |
| 12 | 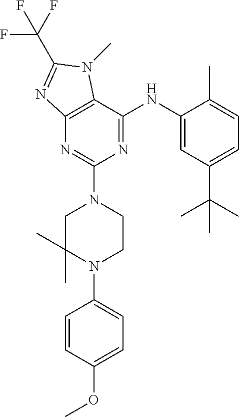 | N-(5-tert-butyl-2- methylphenyl)-2-[4-(4- methoxyphenyl)-3,3- dimethylpiperazin-1-yl]-7- methyl-8-(trifluoromethyl)- 7H-purin-6-amine | Calc'd 582.3, found 582.1 |
| 13 | 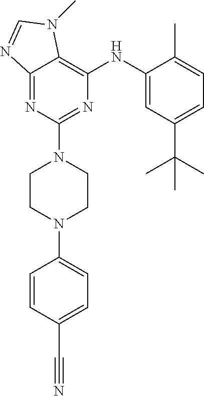 | 4-(4-{6-[(5-tert-butyl-2- methylphenyl)amino]-7- methyl-7H-purin-2- yl}piperazin-1-yl)benzonitrile | Calc'd 481.3, found 481.1 |
| 14 | 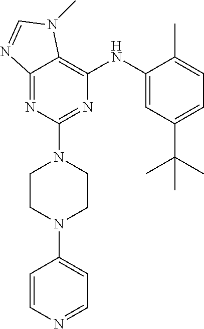 | N-(5-tert-butyl-2- methylphenyl)-7-methyl-2-(4- pyridin-4-ylpiperazin-1-yl)- 7H-purin-6-amine | Calc'd 457.3, found 457.1 |
| 15 | 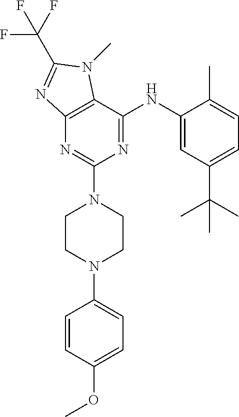 | N-(5-tert-butyl-2- methylphenyl)-2-[4-(4- methoxyphenyl)piperazin-1- yl]-7-methyl-8- (trifluoromethyl)-7H-purin-6- amine | Calc'd 554.3, found 554.1 |
| 16 | 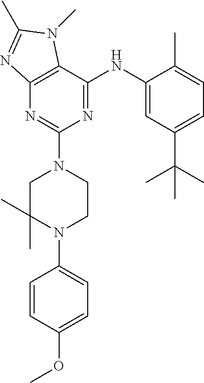 | N-(5-tert-butyl-2- methylphenyl)-2-[4-(4- methoxyphenyl)-3,3- dimethylpiperazin-1-yl]-7,8- dimethyl-7H-purin-6-amine | Calc'd 528.3, found 528.2 |
| 17 | 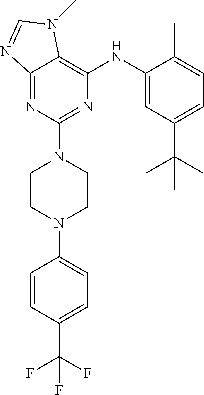 | N-(5-tert-butyl-2- methylphenyl)-7-methyl-2- {4-[4-(trifluoromethyl) phenyl]piperazin-1-yl}- 7H-purin-6-amine | Calc'd 524.3, found 524.2 |
| 18 | 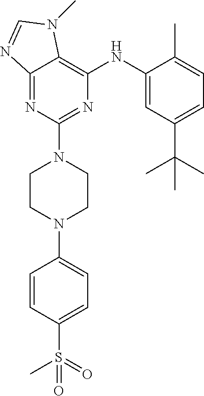 | N-(5-tert-butyl-2- methylphenyl)-7-methyl- 2-{4-[4-(methylsulfonyl) phenyl]piperazin-1-yl}-7H- purin-6-amine | Calc'd 534.3, found 534.2 |
| 19 | 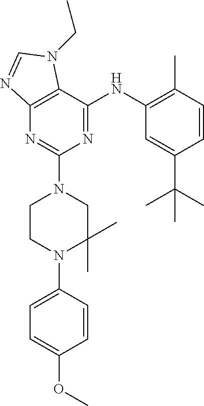 | N-(5-tert-butyl-2- methylphenyl)-7-ethyl-2-[4-(4- methoxyphenyl)-3,3- dimethylpiperazin-1-yl]-7H- purin-6-amine | Calc'd 528.3, found 528.2 |
| 20 | 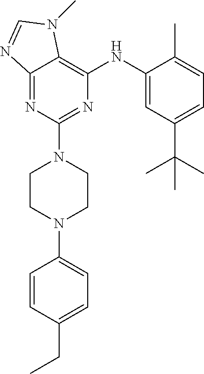 | N-(5-tert-butyl-2- methylphenyl)-2-[4-(4- ethylphenyl)piperazin-1-yl]-7- methyl-7H-purin-6-amine | Calc'd 484.3, found 484.3 |
| 21 | 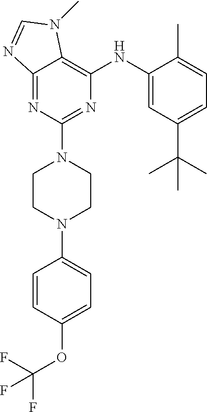 | N-(5-tert-butyl-2- methylphenyl)-7-methyl-2- {4-[4-(trifluoromethoxy) phenyl]piperazin-1-yl}- 7H-purin-6-amine | Calc'd 540.3, found 540.2 |
| 22 | 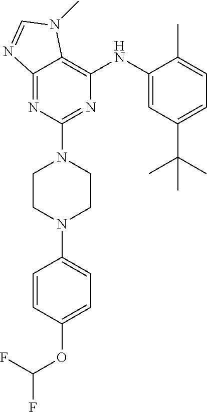 | N-(5-tert-butyl-2- methylphenyl)-2-{4-[4- (difluoromethoxy)phenyl] piperazin-1-yl}-7-methyl-7H- purin-6-amine | Calc'd 522.3, found 522.2 |
| 23 | 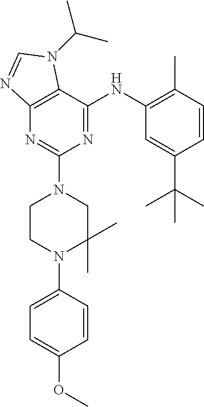 | N-(5-tert-butyl-2- methylphenyl)-7-isopropyl-2- [4-(4-methoxyphenyl)-3,3- dimethylpiperazin-1-yl]-7H- purin-6-amine | Calc'd 542.4, found 542.2 |
| 24 | 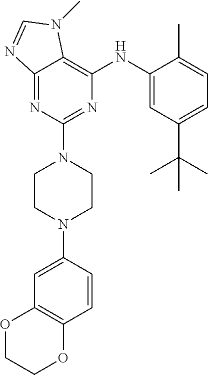 | N-(5-tert-butyl-2- methylphenyl)-2-[4-(2,3- dihydro-1,4-benzodioxin-6- yl)piperazin-1-yl]-7-methyl- 7H-purin-6-amine | Calc'd 514.3, found 514.3 |
| 25 | 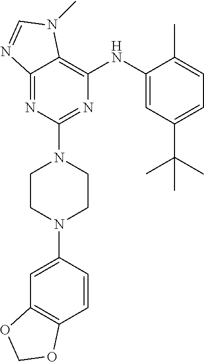 | 2-[4-(1,3-benzodioxol-5- yl)piperazin-1-yl]-N-(5-tert- butyl-2-methylphenyl)-7- methyl-7H-purin-6-amine | Calc'd 500.3, found 500.2 |
| 26 | 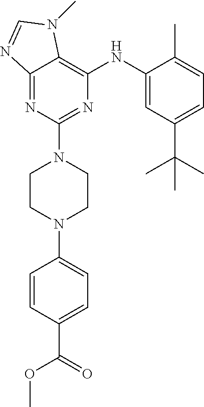 | methyl 4-(4-{6-[(5-tert-butyl- 2-methylphenyl)amino]-7- methyl-7H-purin-2- yl}piperazin-1-yl)benzoate | Calc'd 514.3, found 514.3 |
| 27 | 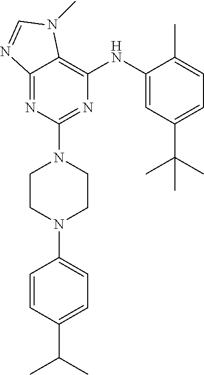 | N-(5-tert-butyl-2- methylphenyl)-2-[4-(4- isopropylphenyl)piperazin-1- yl]-7-methyl-7H-purin-6- amine | Calc'd 498.3, found 498.3 |
| 28 | 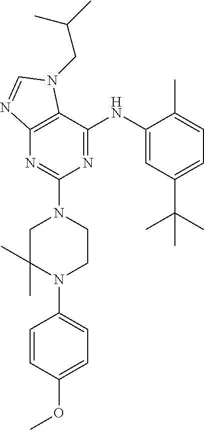 | N-(5-tert-butyl-2- methylphenyl)-7-isobutyl-2-[4- (4-methoxyphenyl)-3,3- dimethylpiperazin-1-yl]-7H- purin-6-amine | Calc'd 556.4, found 556.3 |
| 29 | 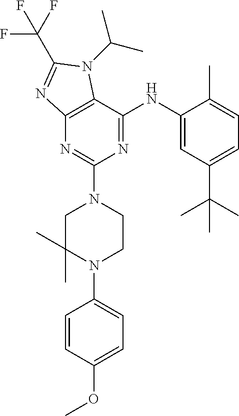 | N-(5-tert-butyl-2- methylphenyl)-7-isopropyl-2- [4-(4-methoxyphenyl)-3,3- dimethylpiperazin-1-yl]-8- (trifluoromethyl)-7H-purin-6- amine | Calc'd 610.3, found 610.3 |
| 30 | 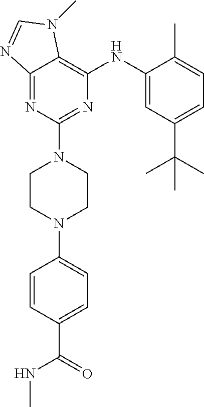 | 4-(4-{6-[(5-tert-butyl-2- methylphenyl)amino]-7- methyl-7H-purin-2- yl}piperazin-1-yl)-N- methylbenzamide | Calc'd 513.3, found 513.2 |
| 31 | 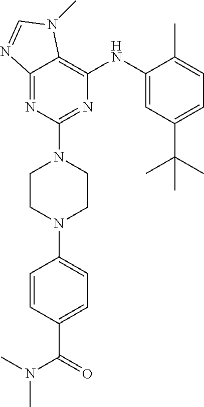 | 4-(4-{6-[(5-tert-butyl-2- methylphenyl)amino]-7- methyl-7H-purin-2- yl}piperazin-1-yl)-N,N- dimethylbenzamide | Calc'd 527.3, found 527.3 |
| 32 | 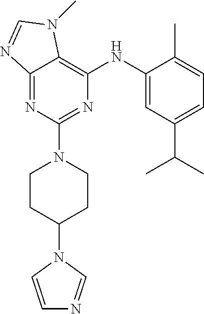 | 2-[4-(1H-imidazol-1- yl)piperidin-1-yl]-N-(5- isopropyl-2-methylphenyl)-7- methyl-7H-purin-6-amine | Calc'd 431.3, found 431.2 |
| 33 | 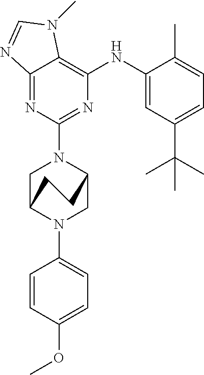 | N-(5-tert-butyl-2- methylphenyl)-2-[(1S,4S)-5- (4-methoxyphenyl)-2,5- diazabicyclo[2.2.2]oct-2-yl]- 7-methyl-7H-purin-6-amine | Calc'd 512.3, found 512.2 |
| 34 | 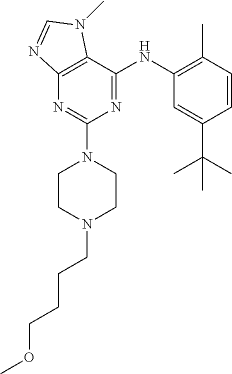 | N-(5-tert-butyl-2- methylphenyl)-2-[4-(4- methoxybutyl)piperazin-1-yl]- 7-methyl-7H-purin-6-amine | Calc'd 466.3, found 466.3 |
| 35 | 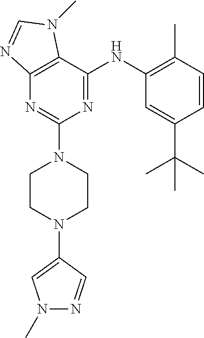 | N-(5-tert-butyl-2- methylphenyl)-7-methyl-2-[4- (1-methyl-1H-pyrazol-4- yl)piperazin-1-yl]-7H-purin-6- amine | Calc'd 460.3, found 460.2 |
| 36 | 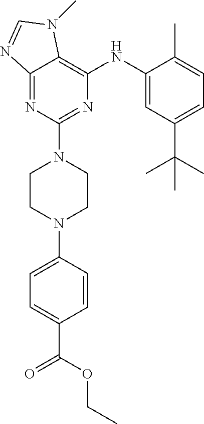 | ethyl 4-(4-{6-[(5-tert-butyl-2- methylphenyl)amino]-7- methyl-7H-purin-2- yl}piperazin-1-yl)benzoate | Calc'd 528.3, found 528.2 |
| 37 | 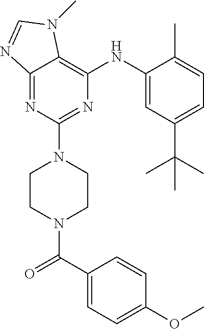 | N-(5-tert-butyl-2- methylphenyl)-2-[4-(4- methoxybenzoyl)piperazin-1- yl]-7-methyl-7H-purin-6-amine | Calc'd 514.3, found 514.2 |
| 38 | 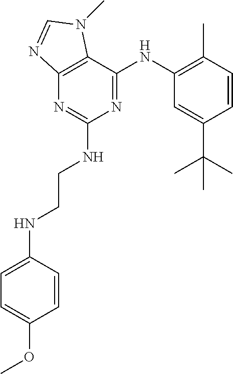 | N~6~-(5-tert-butyl-2- methylphenyl)-N~2~-{2-[(4- methoxyphenyl)amino]ethyl}- 7-methyl-7H-purine-2,6- diamine | Calc'd 460.3, found 460.2 |
| 39 | 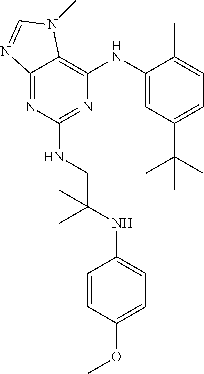 | N~6~-(5-tert-butyl-2- methylphenyl)-N~2~-{2-[(4- methoxyphenyl)amino]-2- methylpropyl}-7-methyl-7H- purine-2,6-diamine | Calc'd 488.3, found 488.2 |
| 40 | 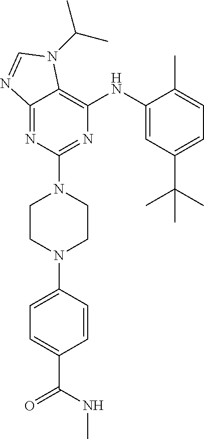 | 4-(4-{6-[(5-tert-butyl-2- methylphenyl)amino]-7- isopropyl-7H-purin-2- yl}piperazin-1-yl)-N- methylbenzamide | Calc'd 541.3, found 541.3 |
| 41 | 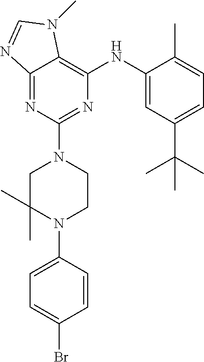 | 2-[4-(4-bromophenyl)-3,3- dimethylpiperazin-1-yl]-N-(5- tert-butyl-2-methylphenyl)-7- methyl-7H-purin-6-amine | Calc'd 562.2, found 562.2 |
| 42 | 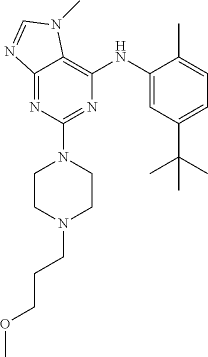 | N-(5-tert-butyl-2- methylphenyl)-2-[4-(3- methoxypropyl)piperazin-1- yl]-7-methyl-7H-purin-6-amine | Calc'd 452.3, found 452.2 |
| 43 | 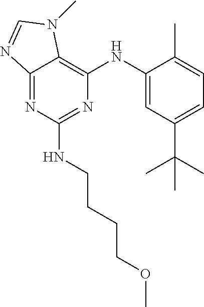 | N~6~-(5-tert-butyl-2- methylphenyl)-N~2~-(4- methoxybutyl)-7-methyl-7H- purine-2,6-diamine | Calc'd 397.3, found 397.3 |
| 44 | 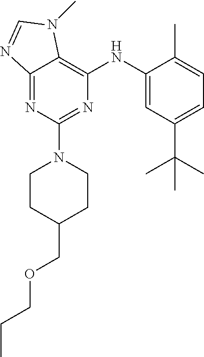 | N-(5-tert-butyl-2- methylphenyl)-7-methyl-2-[4- (propoxymethyl)piperidin-1- yl]-7H-purin-6-amine | Calc'd 451.3, found 451.3 |
| 45 | 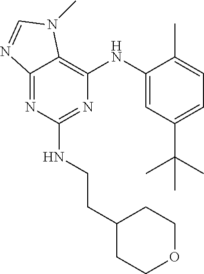 | N~6~-(5-tert-butyl-2- methylphenyl)-7-methyl- N~2~-[2-(tetrahydro-2H- pyran-4-yl)ethyl]-7H-purine- 2,6-diamine | Calc'd 423.3, found 423.3 |
| 46 | 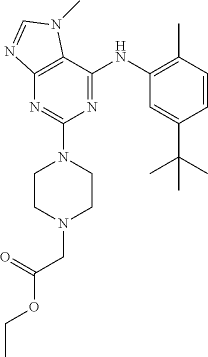 | ethyl (4-{6-[(5-tert-butyl-2- methylphenyl)amino]-7- methyl-7H-purin-2- yl}piperazin-1-yl)-acetate | Calc'd 466.3, found 466.2 |
| 47 | 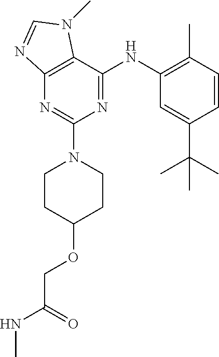 | 2-[(1-{6-[(5-tert-butyl-2- methylphenyl)amino]-7- methyl-7H-purin-2- yl}piperidin-4-yl)oxy]-N- methylacetamide | Calc'd 466.3, found 466.2 |
| 48 | 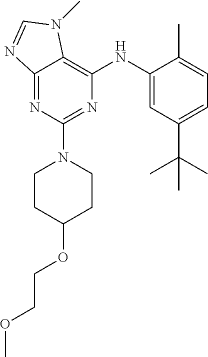 | N-(5-tert-butyl-2- methylphenyl)-2-[4-(2- methoxyethoxy)piperidin-1- yl]-7-methyl-7H-purin-6- amine | Calc'd 453.3, found 453.2 |
| 49 | 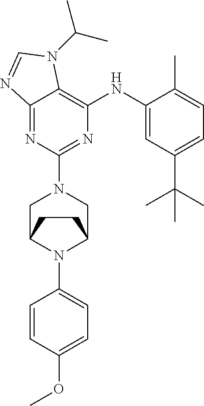 | N-(5-tert-butyl-2- methylphenyl)-7-isopropyl-2- [(1R,5S)-8-(4- methoxyphenyl)-3,8- diazabicyclo[3.2.1]oct-3-yl]- 7H-purin-6-amine | Calc'd 540.3, found 540.3 |
| 50 | 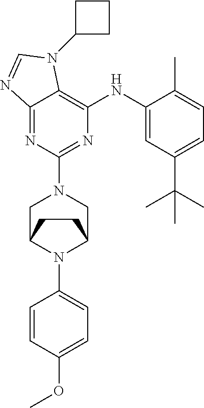 | N-(5-tert-butyl-2- methylphenyl)-7-cyclobutyl-2- [(1R,5S)-8-(4- methoxyphenyl)-3,8- diazabicyclo[3.2.1]oct-3-yl]- 7H-purin-6-amine | Calc'd 552.3, found 552.3 |
| 51 | 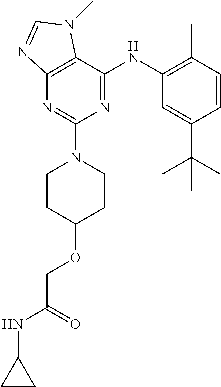 | 2-[(1-{6-[(5-tert-butyl-2- methylphenyl)amino]-7- methyl-7H-purin-2- yl}piperidin-4-yl)oxy]-N- cyclopropylacetamide | Calc'd 492.3, found 492.2 |
| 52 | 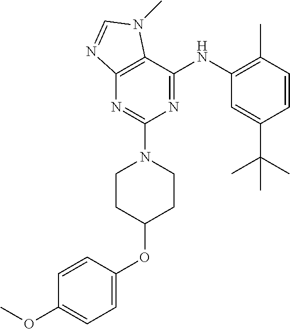 | N-(5-tert-butyl-2- methylphenyl)-2-[4-(4- methoxyphenoxy)piperidin-1- yl]-7-methyl-7H-purin-6- amine | Calc'd 501.3, found 501.2 |
| 53 | 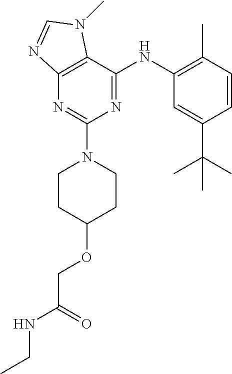 | 2-[(1-{6-[(5-tert-butyl-2- methylphenyl)amino]-7- methyl-7H-purin-2- yl}piperidin-4-yl)oxy]-N- ethylacetamide | Calc'd 480.3, found 480.2 |
| 54 | 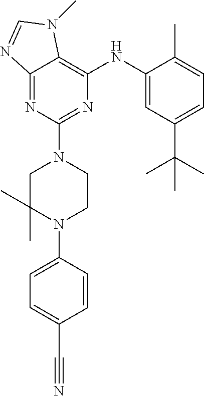 | 4-(4-{6-[(5-tert-butyl-2- methylphenyl)amino]-7- methyl-7H-purin-2-yl}-2,2- dimethylpiperazin-1- yl)benzonitrile | Calc'd 509.3, found 509.2 |
| 55 | 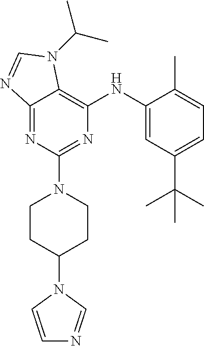 | N-(5-tert-butyl-2- methylphenyl)-2-[4-(1H- imidazol-1-yl)piperidin-1-yl]- 7-isopropyl-7H-purin-6-amine | Calc'd 473.3, found 473.2 |
| 56 | 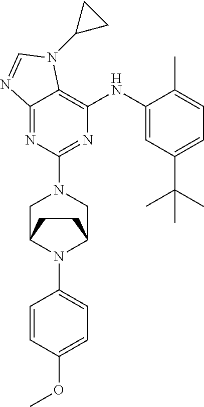 | N-(5-tert-butyl-2- methylphenyl)-7-cyclopropyl- 2-[(1R,5S)-8-(4- methoxyphenyl)-3,8- diazabicyclo[3.2.1]oct-3-yl]- 7H-purin-6-amine | Calc'd 538.3, found 538.2 |
| 57 | 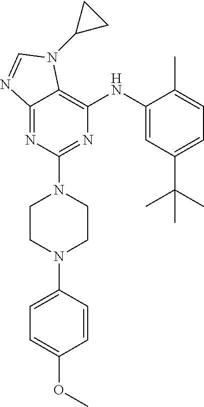 | N-(5-tert-butyl-2- methylphenyl)-7-cyclopropyl- 2-[4-(4-methoxyphenyl) piperazin-1-yl]-7H-purin-6-amine | Calc'd 512.3, found 512.2 |
| 58 | 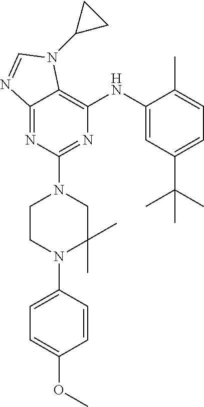 | N-(5-tert-butyl-2- methylphenyl)-7-cyclopropyl- 2-[4-(4-methoxyphenyl)-3,3- dimethylpiperazin-1-yl]-7H- purin-6-amine | Calc'd 540.3, found 540.3 |
| 59 | 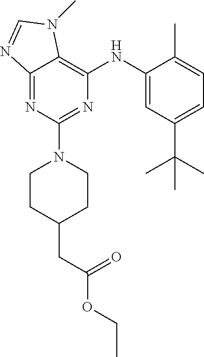 | ethyl (1-{6-[(5-tert-butyl-2- methylphenyl)amino]-7- methyl-7H-purin-2- yl}piperidin-4-yl)acetate | Calc'd 465.3, found 465.2 |
| 60 | 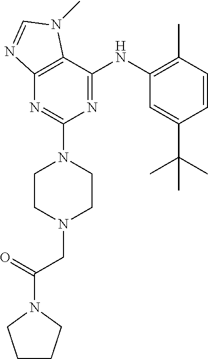 | N-(5-tert-butyl-2- methylphenyl)-7-methyl-2-[4- (2-oxo-2-pyrrolidin-1- ylethyl)piperazin-1-yl]-7H- purin-6-amine | Calc'd 491.3, found 491.2 |
| 61 | 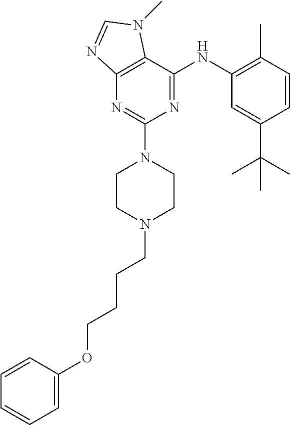 | N-(5-tert-butyl-2- methylphenyl)-7-methyl-2-[4- (4-phenoxybutyl)piperazin-1- yl]-7H-purin-6-amine | Calc'd 528.3, found 528.2 |
| 62 | 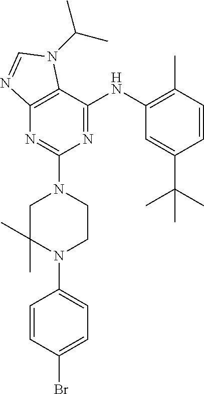 | 2-[4-(4-bromophenyl)-3,3- dimethylpiperazin-1-yl]-N-(5- tert-butyl-2-methylphenyl)-7- isopropyl-7H-purin-6-amine | Calc'd 590.3, found 590.2 |
| 63 | 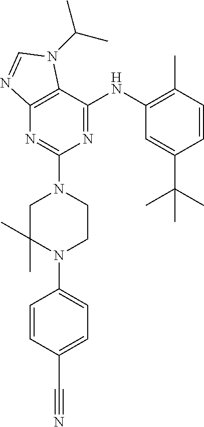 | 4-(4-{6-[(5-tert-butyl-2- methylphenyl)amino]-7- isopropyl-7H-purin-2-yl}-2,2- dimethylpiperazin-1- yl)benzonitrile | Calc'd 537.3, found 537.3 |
| 64 | 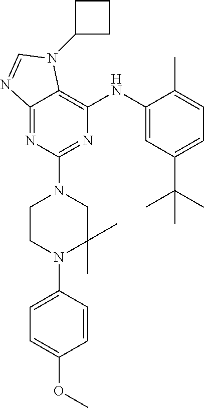 | N-(5-tert-butyl-2- methylphenyl)-7-cyclobutyl-2- [4-(4-methoxyphenyl)-3,3- dimethylpiperazin-1-yl]-7H- purin-6-amine | Calc'd 554.4, found 554.3 |
| 65 | 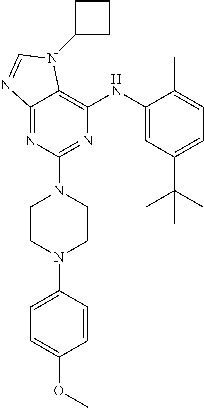 | N-(5-tert-butyl-2- methylphenyl)-7-cyclobutyl-2- [4-(4-methoxyphenyl) piperazin-1-yl]-7H-purin-6-amine | Calc'd 526.3, found 526.2 |
| 66 | 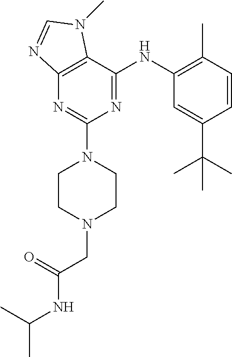 | 2-(4-{6-[(5-tert-butyl-2- methylphenyl)amino]-7- methyl-7H-purin-2- yl}piperazin-1-yl)-N- isopropylacetamide | Calc'd 479.3, found 479.3 |
| 67 | 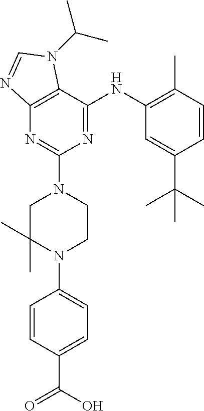 | 4-(4-{6-[(5-tert-butyl-2- methylphenyl)amino]-7- isopropyl-7H-purin-2-yl}-2,2- dimethylpiperazin-1-yl) benzoic acid | Calc'd 556.3, found 556.3 |
| 68 | 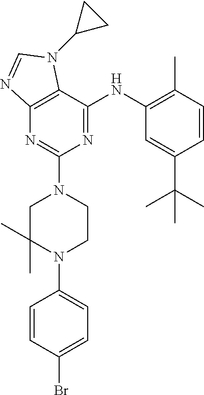 | 2-[4-(4-bromophenyl)-3,3- dimethylpiperazin-1-yl]-N-(5- tert-butyl-2-methylphenyl)-7- cyclopropyl-7H-purin-6-amine | Calc'd 588.2, found 588.2 |
| 69 | 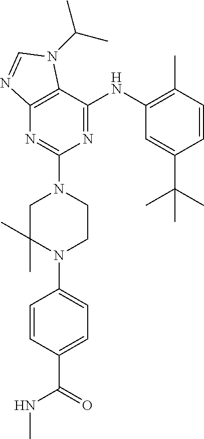 | 4-(4-{6-[(5-tert-butyl-2- methylphenyl)amino]-7- isopropyl-7H-purin-2-yl}-2,2- dimethylpiperazin-1-yl)-N- methylbenzamide | Calc'd 569.4, found 569.3 |
| 70 | 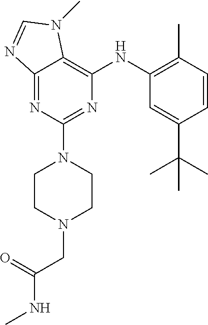 | 2-(4-{6-[(5-tert-butyl-2- methylphenyl)amino]-7- methyl-7H-purin-2- yl}piperazin-1-yl)-N- methylacetamide | Calc'd 451.3, found 451.2 |
| 71 | 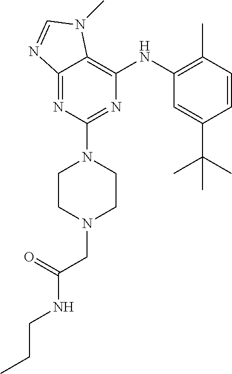 | 2-(4-{6-[(5-tert-butyl-2- methylphenyl)amino]-7- methyl-7H-purin-2- yl}piperazin-1-yl)-N- propylacetamide | Calc'd 479.3, found 479.2 |
| 72 | 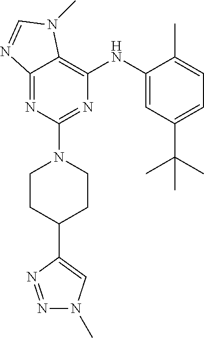 | N-(5-tert-butyl-2- methylphenyl)-7-methyl-2-[4- (1-methyl-1H-1,2,3-triazol-4- yl)piperidin-1-yl]-7H-purin-6- amine | Calc'd 460.3, found 460.2 |
| 73 | 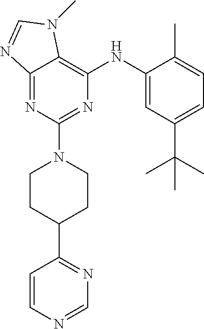 | N-(5-tert-butyl-2- methylphenyl)-7-methyl-2-(4- pyrimidin-4-ylpiperidin-1-yl)- 7H-purin-6-amine | Calc'd 457.3, found 457.2 |
| 74 | 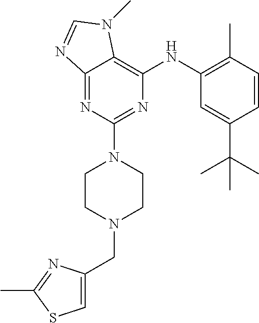 | N-(5-tert-butyl-2- methylphenyl)-7-methyl-2-{4- [(2-methyl-1,3-thiazol-4- yl)methyl]piperazin-1-yl}-7H- purin-6-amine | Calc'd 491.3, found 491.2 |
| 75 | 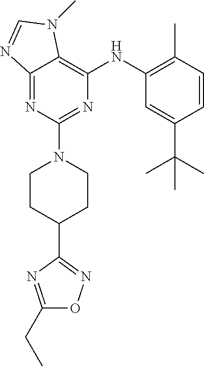 | N-(5-tert-butyl-2- methylphenyl)-2-[4-(5-ethyl- 1,2,4-oxadiazol-3-yl)piperidin- 1-yl]-7-methyl-7H-purin-6-amine | Calc'd 475.3, found 475.0 |
| 76 | 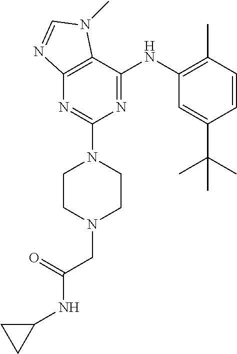 | 2-(4-{6-[(5-tert-butyl-2- methylphenyl)amino]-7- methyl-7H-purin-2- yl}piperazin-1-yl)-N- cyclopropylacetamide | Calc'd 477.3, found 477.2 |
| 77 | 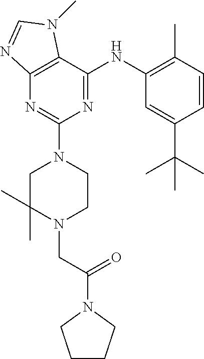 | N-(5-tert-butyl-2- methylphenyl)-2-[3,3- dimethyl-4-(2-oxo-2- pyrrolidin-1-ylethyl)piperazin- 1-yl]-7-methyl-7H-purin-6-amine | Calc'd 519.4, found 519.3 |
| 78 | 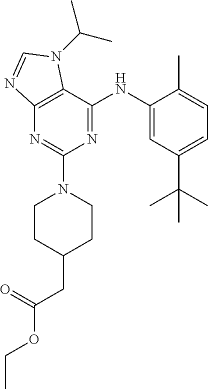 | ethyl (1-{6-[(5-tert-butyl-2- methylphenyl)amino]-7- isopropyl-7H-purin-2- yl}piperidin-4-yl)acetate | Calc'd 493.3, found 493.3 |
| 79 | 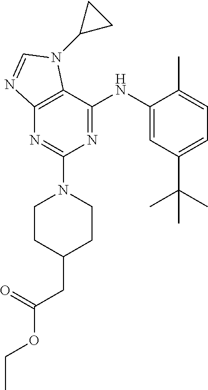 | ethyl (1-{6-[(5-tert-butyl-2- methylphenyl)amino]-7- cyclopropyl-7H-purin-2- yl}piperidin-4-yl)acetate | Calc'd 491.3, found 491.2 |
| 80 | 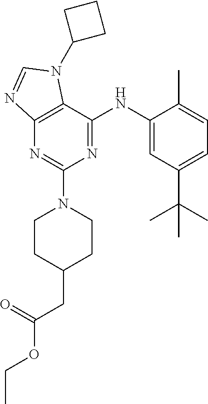 | ethyl (1-{6-[(5-tert-butyl-2- methylphenyl)amino]-7- cyclobutyl-7H-purin-2- yl}piperidin-4-yl)acetate | Calc'd 505.3, found 505.2 |
| 81 | 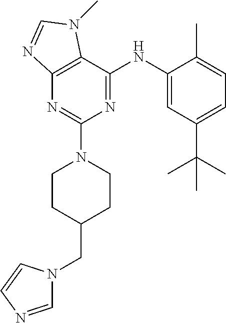 | N-(5-tert-butyl-2- methylphenyl)-2-[4-(1H- imidazol-1-ylmethyl)piperidin- 1-yl]-7-methyl-7H-purin-6-amine | Calc'd 459.3, found 459.2 |
| 82 | 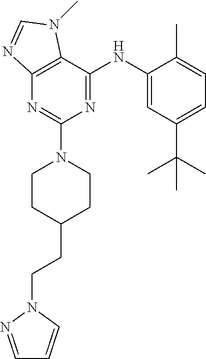 | N-(5-tert-butyl-2- methylphenyl)-7-methyl-2-{4- [2-(1H-pyrazol-1- yl)ethyl]piperidin-1-yl}-7H- purin-6-amine | Calc'd 473.3, found 473.3 |
| 83 | 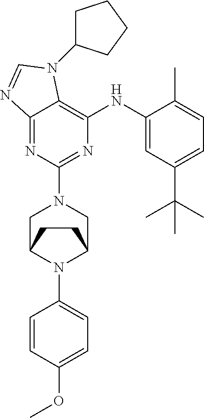 | N-(5-tert-butyl-2- methylphenyl)-7-cyclopentyl- 2-[(1R,5S)-8-(4- methoxyphenyl)-3,8- diazabicyclo[3.2.1]oct-3-yl]- 7H-purin-6-amine | Calc'd 566.4, found 566.3 |
| 84 | 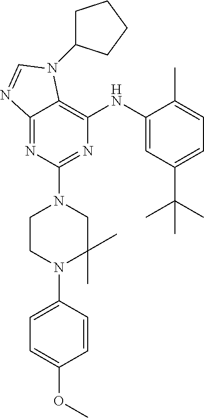 | N-(5-tert-butyl-2- methylphenyl)-7-cyclopentyl- 2-[4-(4-methoxyphenyl)-3,3- dimethylpiperazin-1-yl]-7H- purin-6-amine | Calc'd 568.4, found 568.3 |
| 85 | 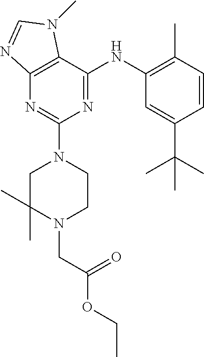 | ethyl (4-{6-[(5-tert-butyl-2- methylphenyl)amino]-7- methyl-7H-purin-2-yl}-2,2- dimethylpiperazin-1-yl)acetate | Calc'd 494.3, found 494.3 |
| 86 | 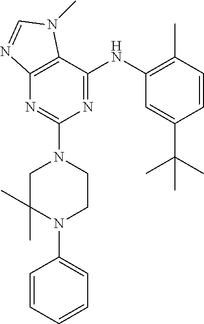 | N-(5-tert-butyl-2- methylphenyl)-2-(3,3- dimethyl-4-phenylpiperazin-1- yl)-7-methyl-7H-purin-6-amine | Calc'd 484.3, found 484.2 |
| 87 | 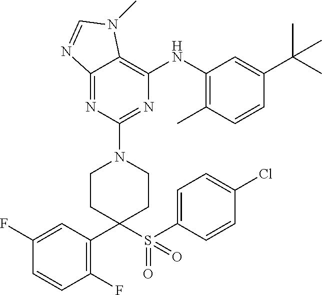 | N-(5-tert-butyl-2- methylphenyl)-2-{4-[(4- chlorophenyl)sulfonyl]-4-(2,5- difluorophenyl)piperidin-1-yl}- 7-methyl-7H-purin-6-amine | Calc'd 665.2, found 665.2 |
| 88 | 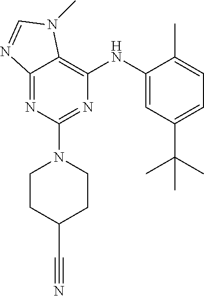 | 1-{6-[(5-tert-butyl-2- methylphenyl)amino]-7- methyl-7H-purin-2- yl}piperidine-4-carbonitrile | Calc'd 404.3, found 404.2 |
| 89 | 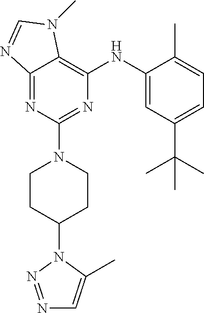 | N-(5-tert-butyl-2- methylphenyl)-7-methyl-2-[4- (5-methyl-1H-1,2,3-triazol-1- yl)piperidin-1-yl]-7H-purin-6- amine | Calc'd 460.3, found 460.2 |
| 90 | 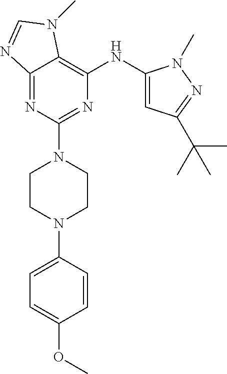 | N-(3-tert-butyl-1-methyl-1H- pyrazol-5-yl)-2-[4-(4- methoxyphenyl)piperazin-1- yl]-7-methyl-7H-purin-6-amine | Calc'd 476.3, found 476.3 |
| 91 | 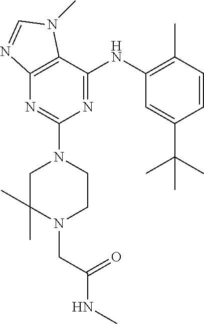 | 2-(4-{6-[(5-tert-butyl-2- methylphenyl)amino]-7- methyl-7H-purin-2-yl}-2,2- dimethylpiperazin-1-yl)-N- methylacetamide | Calc'd 479.3, found 479.3 |
| 92 | 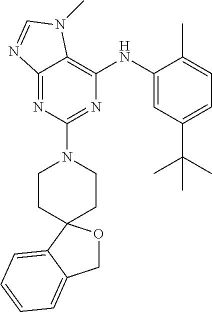 | N-(5-tert-butyl-2- methylphenyl)-7-methyl-2- (1′H,3H-spiro[2-benzofuran- 1,4′-piperidin]-1-yl)-7H-purin- 6-amine | Calc'd 483.3, found 481.3 |
| 93 | 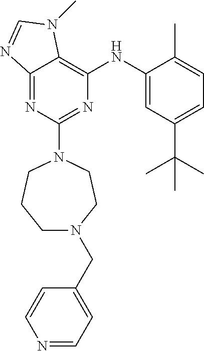 | N-(5-tert-butyl-2- methylphenyl)-7-methyl-2-[4- (pyridin-4-ylmethyl)-1,4- diazepan-1-yl]-7H-purin-6- amine | Calc'd 485.3, found 485.3 |
| 94 | 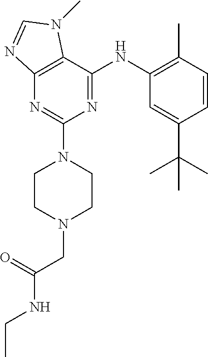 | 2-(4-{6-[(5-tert-butyl-2- methylphenyl)amino]-7- methyl-7H-purin-2- yl}piperazin-1-yl)-N- ethylacetamide | Calc'd 465.3, found 465.2 |
| 95 | 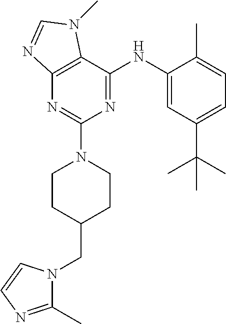 | N-(5-tert-butyl-2- methylphenyl)-7-methyl-2-{4- [(2-methyl-1H-imidazol-1- yl)methyl]piperidin-1-yl}-7H- purin-6-amine | Calc'd 473.3, found 473.3 |
| 96 | 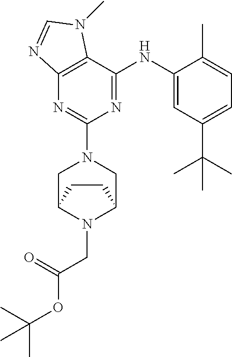 | tert-butyl [(1R,5S)-3-{6-[(5- tert-butyl-2- methylphenyl)amino]-7- methyl-7H-purin-2-yl}-3,8- diazabicyclo[3.2.1]oct-8- yl]acetate | Calc'd 520.3, found 520.3 |
| 97 | 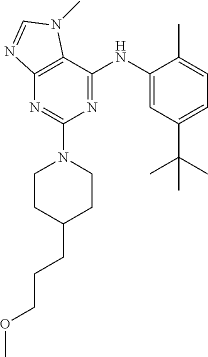 | N-(5-tert-butyl-2- methylphenyl)-2-[4-(3- methoxypropyl)piperidin-1-yl]- 7-methyl-7H-purin-6-amine | Calc'd 451.3, found 451.2 |
| 98 | 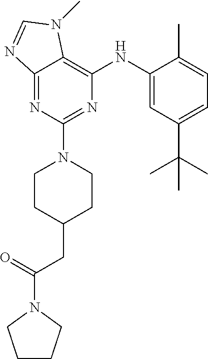 | N-(5-tert-butyl-2- methylphenyl)-7-methyl-2-[4- (2-oxo-2-pyrrolidin-1- ylethyl)piperidin-1-yl]-7H- purin-6-amine | Calc'd 490.3, found 490.3 |
| 99 | 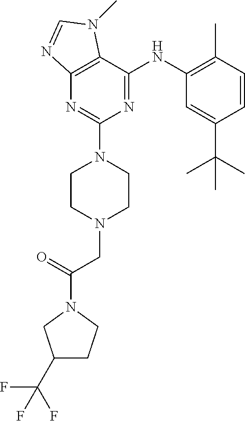 | N-(5-tert-butyl-2- methylphenyl)-7-methyl-2-(4- {2-oxo-2-[3- (trifluoromethyl)pyrrolidin-1- yl]ethyl}piperazin-1-yl)-7H- purin-6-amine | Calc'd 559.3, found 559.3 |
| 100 | 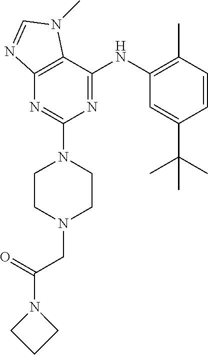 | 2-[4-(2-azetidin-1-yl-2- oxoethyl)piperazin-1-yl]-N-(5- tert-butyl-2-methylphenyl)-7- methyl-7H-purin-6-amine | Calc'd 477.3, found 477.3 |
| 101 | 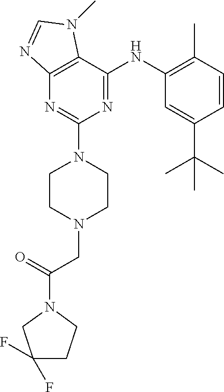 | N-(5-tert-butyl-2- methylphenyl)-2-{4-[2-(3,3- difluoropyrrolidin-1-yl)-2- oxoethyl]piperazin-1-yl}-7- methyl-7H-purin-6-amine | Calc'd 527.3, found 527.2 |
| 102 | 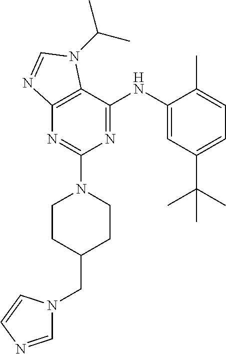 | N-(5-tert-butyl-2- methylphenyl)-2-[4-(1H- imidazol-1-ylmethyl)piperidin- 1-yl]-7-isopropyl-7H-purin-6- amine | Calc'd 487.3, found 487.3 |
| 103 | 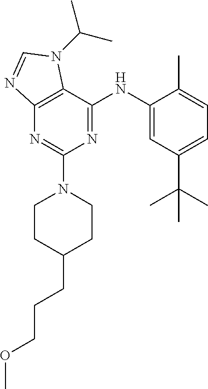 | N-(5-tert-butyl-2- methylphenyl)-7-isopropyl-2- [4-(3-methoxypropyl)piperidin- 1-yl]-7H-purin-6-amine | Calc'd 479.3, found 479.3 |
| 104 | 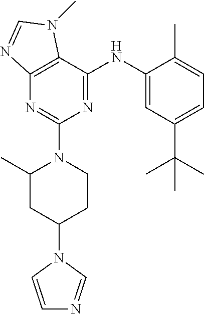 | N-(5-tert-butyl-2- methylphenyl)-2-[4-(1H- imidazol-1-yl)-2- methylpiperidin-1-yl]-7- methyl-7H-purin-6-amine | Calc'd 459.3, found 459.2 |
| 105 | 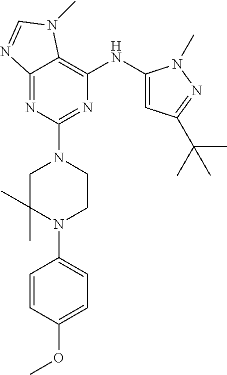 | N-(3-tert-butyl-1-methyl-1H- pyrazol-5-yl)-2-[4-(4- methoxyphenyl)-3,3- dimethylpiperazin-1-yl]-7- methyl-7H-purin-6-amine | Calc'd 504.3, found 504.3 |
| 106 | 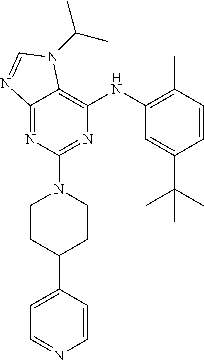 | N-(5-tert-butyl-2- methylphenyl)-7-isopropyl-2- (4-pyridin-4-ylpiperidin-1-yl)- 7H-purin-6-amine | Calc'd 484.3, found 485.3 |
| 107 | 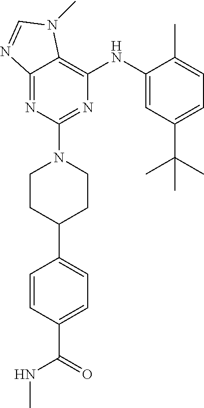 | 4-(1-{6-[(5-tert-butyl-2- methylphenyl)amino]-7- methyl-7H-purin-2- yl}piperidin-4-yl)-N- methylbenzamide | Calc'd 512.3, found 512.3 |
| 108 | 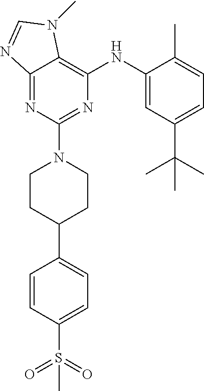 | N-(5-tert-butyl-2- methylphenyl)-7-methyl-2-{4- [4-(methylsulfonyl)phenyl] piperidin-1-yl}-7H-purin-6- amine | Calc'd 533.3, found 533.2 |
| 109 | 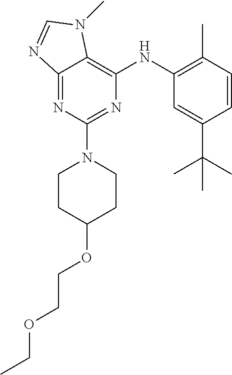 | N-(5-tert-butyl-2- methylphenyl)-2-[4-(2- ethoxyethoxy)piperidin-1-yl]- 7-methyl-7H-purin-6-amine | Calc'd 467.3, found 467.1 |
| 110 | 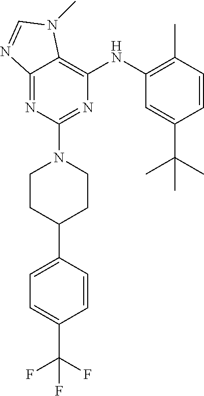 | N-(5-tert-butyl-2- methylphenyl)-7-methyl-2-{4- [4-(trifluoromethyl)phenyl] piperidin-1-yl}-7H-purin-6-amine | Calc'd 523.3, found 523.0 |
| 111 | 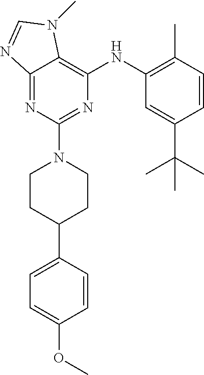 | N-(5-tert-butyl-2- methylphenyl)-2-[4-(4- methoxyphenyl)piperidin-1- yl]-7-methyl-7H-purin-6-amine | Calc'd 485.3, found 485.2 |
| 112 | 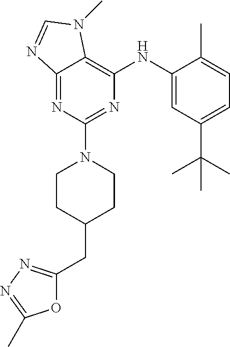 | N-(5-tert-butyl-2- methylphenyl)-7-methyl-2-{4- [(5-methyl-1,3,4-oxadiazol-2- yl)methyl]piperidin-1-yl}-7H- purin-6-amine | Calc'd 475.3, found 475.2 |
| 113 | 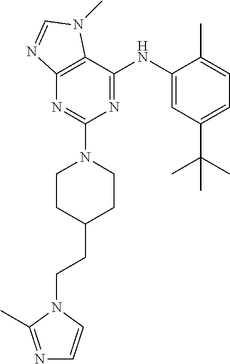 | N-(5-tert-butyl-2- methylphenyl)-7-methyl-2-{4- [2-(2-methyl-1H-imidazol-1- yl)ethyl]piperidin-1-yl}-7H- purin-6-amine | Calc'd 487.3, found 487.3 |
| 114 | 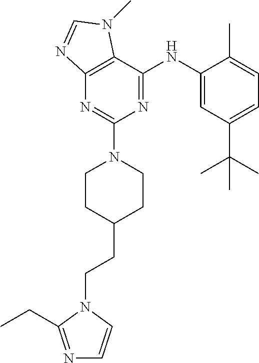 | N-(5-tert-butyl-2- methylphenyl)-2-{4-[2-(2- ethyl-1H-imidazol-1- yl)ethyl]piperidin-1-yl}-7- methyl-7H-purin-6-amine | Calc'd 501.3, found 501.2 |
| 115 | 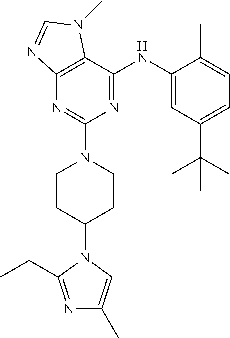 | N-(5-tert-butyl-2- methylphenyl)-2-{4-[2-(2- ethyl-4-methyl-1H-imidazol-1- yl)ethyl]piperidin-1-yl}-7- methyl-7H-purin-6-amine | Calc'd 515.4, found 514.9 |
| 116 | 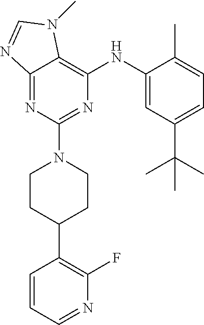 | N-(5-tert-butyl-2- methylpheny)-2-[4-(2- fluoropyridin-3-yl)piperidin-1- yl]-7-methyl-7H-purin-6-amine | Calc'd 474.3, found 474.3 |
| 117 | 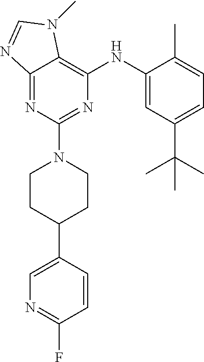 | N-(5-tert-butyl-2- methylphenyl)-2-[4-(6- fluoropyridin-3-yl)piperidin-1- yl]-7-methyl-7H-purin-6-amine | Calc'd 474.3, found 474.3 |
| 118 | 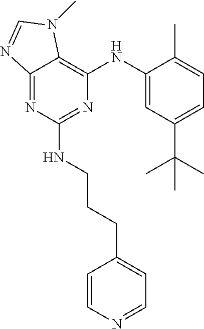 | N~6~-(5-tert-butyl-2- methylphenyl)-7-methyl- N~2~-(3-pyridin-4-ylpropyl)- 7H-purine-2,6-diamine | Calc'd 430.3, found 430.3 |
| 119 | 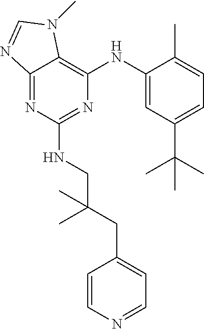 | N~6~-(5-tert-butyl-2- methylphenyl)-N~2~-(2,2- dimethyl-3-pyridin-4- ylpropyl)-7-methyl-7H-purine- 2,6-diamine | Calc'd 458.3, found 458.2 |
| 120 | 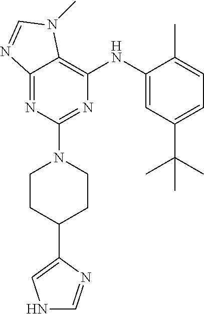 | N-(5-tert-butyl-2- methylphenyl)-2-[4-(1H- imidazol-4-yl)piperidin-1-yl]- 7-methyl-7H-purin-6-amine | Calc'd 445.3, found 445.3 |
| 121 | 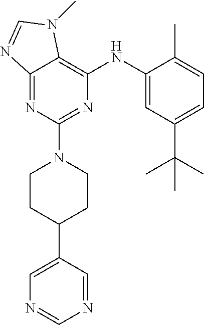 | N-(5-tert-butyl-2- methylphenyl)-7-methyl-2-(4- pyrimidin-5-ylpiperidin-1-yl)- 7H-purin-6-amine | Calc'd 457.3, found 457.3 |
| 122 | 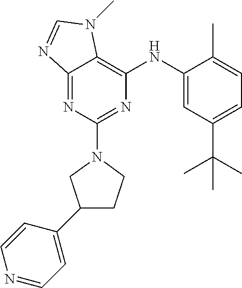 | N-(5-tert-butyl-2- methylphenyl)-7-methyl-2-(3- pyridin-4-ylpyrrolidin-1-yl)- 7H-purin-6-amine | Calc'd 442.3, found 442.2 |
| 123 | 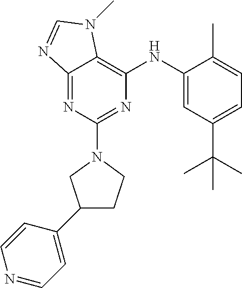 | N-(5-tert-butyl-2- methylphenyl)-7-methyl-2-(3- pyridin-4-ylpyrrolidin-1-yl)- 7H-purin-6-amine | Calc'd 442.3, found 442.3 |
| 124 | 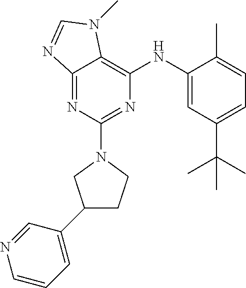 | N-(5-tert-butyl-2- methylphenyl)-7-methyl-2-(3- pyridin-3-ylpyrrolidin-1-yl)- 7H-purin-6-amine | Calc'd 442.3, found 442.3 |
| 125 | 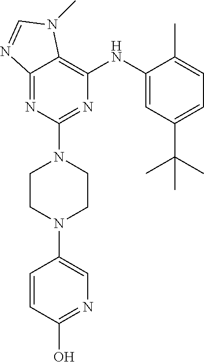 | 5-(4-{6-[(5-tert-butyl-2- methylphenyl)amino]-7- methyl-7H-purin-2- yl}piperazin-1-yl)pyrrolidin-2-ol | Calc'd 473.3, found 473.2 |
| 126 | 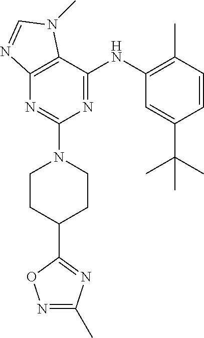 | N-(5-tert-butyl-2- methylphenyl)-7-methyl-2-[4- (3-methyl-1,2,4-oxadiazol-5- yl)piperidin-1-yl]-7H-purin-6- amine | Calc'd 461.3, found 461.2 |
| 127 | 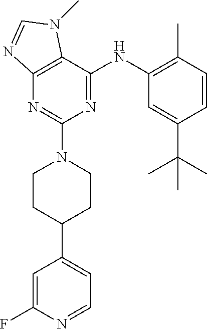 | N-(5-tert-butyl-2- methylphenyl)-2-[4-(2- fluoropyridin-4-yl)piperidin-1- yl]-7-methyl-7H-purin-6-amine | Calc'd 474.3, found 474.2 |
| 128 | 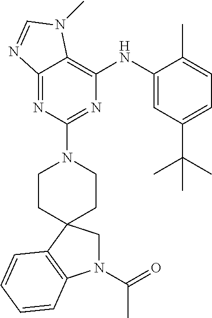 | 2-(1-acetyl-1,2-dihydro-1′H- spiro[indole-3,4′-piperidin]-1′- yl)-N-(5-tert-butyl-2- methylphenyl)-7-methyl-7H- purin-6-amine | Calc'd 524.3, found 524.2 |
| 129 | 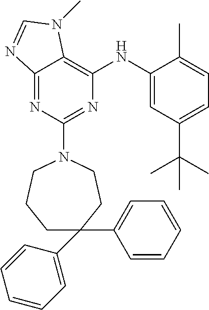 | N-(5-tert-butyl-2- methylphenyl)-2-(4,4- diphenylazepan-1-yl)-7- methyl-7H-purin-6-amine | Calc'd 545.3, found 545.2 |
| 130 | 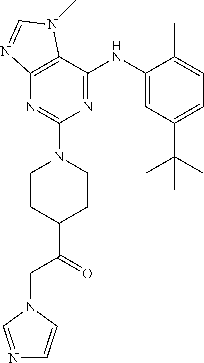 | 1-(1-{6-[(5-tert-butyl-2- methylphenyl)amino]-7- methyl-7H-purin-2- yl}piperidin-4-yl)-2-(1H- imidazol-1-yl)ethanone | Calc'd 487.3, found 487.2 |
| 131 | 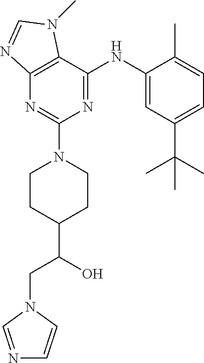 | 1-(1-{6-[(5-tert-butyl-2- methylphenyl)amino]-7- methyl-7H-purin-2- yl}piperidin-4-yl)-2-(1H- imidazol-1-yl)ethanol | Calc'd 489.3, found 489.2 |
| 132 | 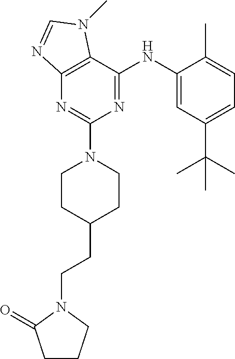 | 1-[2-(1-{6-[(5-tert-butyl-2- methylphenyl)amino]-7- methyl-7H-purin-2- yl}piperidin-4- yl)ethyl]pyrrolidin-2-one | Calc'd 490.3, found 490.3 |
| 133 | 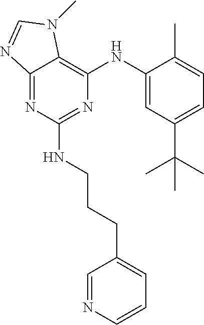 | N~6~-(5-tert-butyl-2- methylphenyl)-7-methyl- N~2~-(3-pyridin-3-ylpropyl)- 7H-purine-2,6-diamine | Calc'd 430.3, found 430.3 |
| 134 | 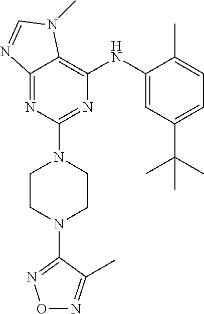 | N-(5-tert-butyl-2- methylphenyl)-7-methyl-2-[4- (4-methyl-1,2,5-oxadiazol-3- yl)piperazin-1-yl]-7H-purin-6- amine | Calc'd 462.3, found 462.3 |
| 135 | 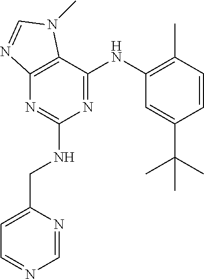 | N~6~-(5-tert-butyl-2- methylphenyl)-7-methyl- N~2~-(pyrimidin-4-ylmethyl)- 7H-purine-2,6-diamine | Calc'd 403.2, found 403.3 |
| 136 | 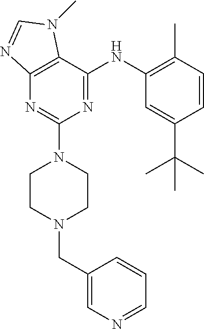 | N-(5-tert-butyl-2- methylphenyl)-7-methyl-2-[4- (pyridin-3-ylmethyl)piperazin- 1-yl]-7H-purin-6-amine | Calc'd 471.3, found 471.3 |
| 137 | 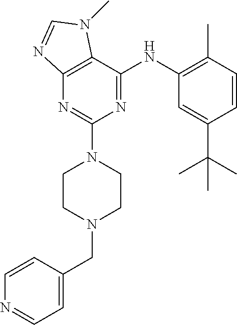 | N-(5-tert-butyl-2- methylphenyl)-7-methyl-2-[4- (pyridin-4-ylmethyl)piperazin- 1-yl]-7H-purin-6-amine | Calc'd 471.3, found 471.3 |
| 138 | 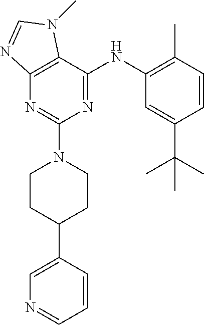 | N-(5-tert-butyl-2- methylphenyl)-7-methyl-2-(4- pyridin-3-ylpiperidin-1-yl)-7H- purin-6-amine | Calc'd 456.3, found 456.3 |
| 139 | 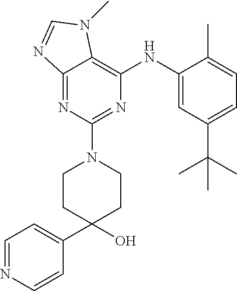 | 1-{6-[(5-tert-butyl-2- methylphenyl)amino]-7- methyl-7H-purin-2-yl}-4- pyridin-4-ylpiperidin-4-ol | Calc'd 472.3, found 472.2 |
| 140 | 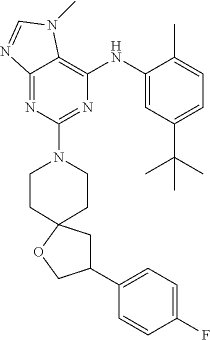 | N-(5-tert-butyl-2- methylphenyl)-2-[3-(4- fluorophenyl)-1-oxa-8- azaspiro[4.5]dec-8-yl]-7- methyl-7H-purin-6-amine | Calc'd 529.3, found 529.3 |
| 141 | 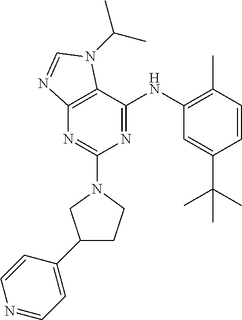 | N-(5-tert-butyl-2- methylphenyl)-7-isopropyl-2- (3-pyridin-4-ylpyrrolidin-1-yl)- 7H-purin-6-amine | Calc'd 470.3, found 470.3 |
| 142 | 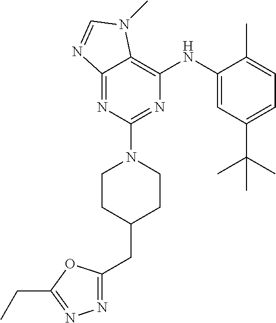 | N-(5-tert-butyl-2- methylphenyl)-2-{4-[(5-ethyl- 1,3,4-oxadiazol-2- yl)methyl]piperidin-1-yl}-7- methyl-7H-purin-6-amine | Calc'd 489.3, found 489.3 |
| 143 | 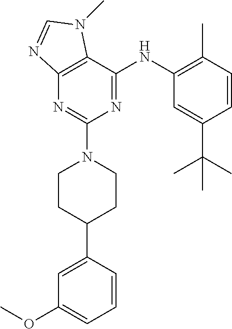 | N-(5-tert-butyl-2- methylphenyl)-2-[4-(3- methoxyphenyl)piperidin-1- yl]-7-methyl-7H-purin-6-amine | Calc'd 485.3, found 485.3 |
| 144 | 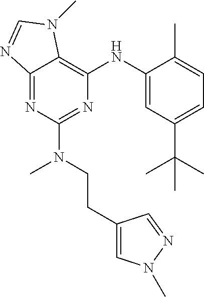 | N~6~-(5-tert-butyl-2- methylphenyl)-N~2~,7- dimethyl-N~2~-[2-(1-methyl- 1H-pyrazol-4-yl)ethyl]-7H- purine-2,6-diamine | Calc'd 433.3, found 433.3 |
| 145 | 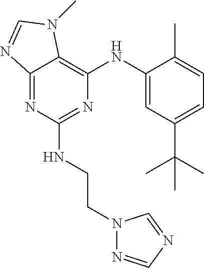 | N~6~-(5-tert-butyl-2- methylphenyl)-7-methyl- N~2~-[2-(1H-1,2,4-triazol-1- yl)ethyl]-7H-purine-2,6- diamine | Calc'd 406.2, found 406.2 |
| 146 | 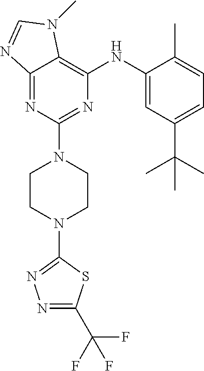 | N-(5-tert-butyl-2- methylphenyl)-7-methyl-2-{4- [5-(trifluoromethyl)-1,3,4- thiadiazol-2-yl]piperazin-1- yl}-7H-purin-6-amine | Calc'd 532.2, found 532.2 |
| 147 | 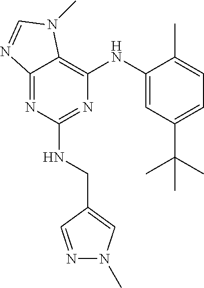 | N~6~-(5-tert-butyl-2- methylphenyl)-7-methyl- N~2~-[(1-methyl-1H-pyrazol- 4-yl)methyl]-7H-purine-2,6- diamine | Calc'd 405.3, found 405.2 |
| 148 | 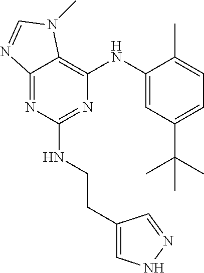 | N~6~-(5-tert-butyl-2- methylphenyl)-7-methyl- N~2~-[2-(1H-pyrazol-4- yl)ethyl]-7H-purine-2,6- diamine | Calc'd 405.3, found 405.2 |
| 149 | 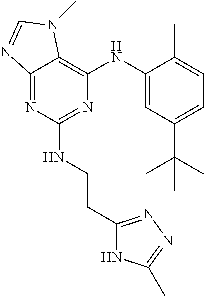 | N~6~-(5-tert-butyl-2- methylphenyl)-7-methyl- N~2~-[2-(5-methyl-4H-1,2,4- triazol-3-yl)ethyl]-7H-purine- 2,6-diamine | Calc'd 420.3, found 420.2 |
| 150 | 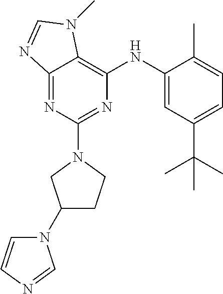 | N-(5-tert-butyl-2- methylphenyl)-2-[3-(1H- imidazol-1-yl)pyrrolidin-1-yl]- 7-methyl-7H-purin-6-amine | Calc'd 431.3, found 431.3 |
| 151 | 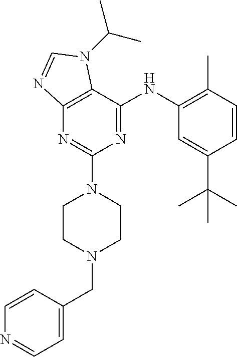 | N-(5-tert-butyl-2- methylphenyl)-7-isopropyl-2- [4-(pyridin-4- ylmethyl)piperazin-1-yl]-7H- purin-6-amine | Calc'd 499.3, found 499.3 |
| 152 | 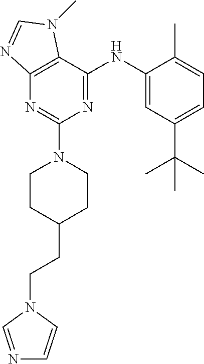 | N-(5-tert-butyl-2- methylphenyl)-2-{4-[2-(1H- imidazol-1-yl)ethyl]piperidin- 1-yl}-7-methyl-7H-purin-6- amine | Calc'd 473.3, found 473.3 |
| 153 | 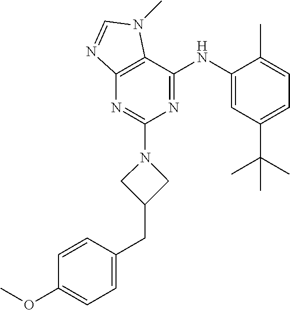 | N-(5-tert-butyl-2- methylphenyl)-2-[3-(4- methoxybenzyl)azetidin-1-yl]- 7-methyl-7H-purin-6-amine | Calc'd 471.3, found 471.3 |
| 154 | 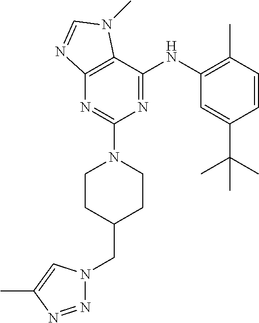 | N-(5-tert-butyl-2- methylphenyl)-7-methyl-2-{4- [(4-methyl-1H-1,2,3-triazol-1- yl)methyl]piperidin-1-yl}-7H- purin-6-amine | Calc'd 474.3, found 474.3 |
| 155 | 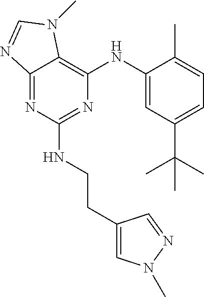 | N~6~-(5-tert-butyl-2- methylphenyl)-7-methyl- N~2~-[2-(1-methyl-1H- pyrazol-4-yl)ethyl]-7H-purine- 2,6-diamine | Calc'd 419.3, found 419.2 |
| 156 | 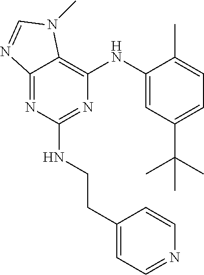 | N~6~-(5-tert-butyl-2- methylphenyl)-7-methyl- N~2~-(2-pyridin-4-ylethyl)- 7H-purine-2,6-diamine | Calc'd 416.3, found 416.2 |
| 157 | 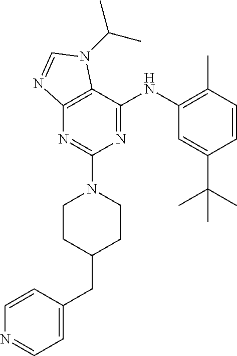 | N-(5-tert-butyl-2- methylphenyl)-7-isopropyl-2- [4-(pyridin-4- ylmethyl)piperidin-1-yl]-7H- purin-6-amine | Calc'd 498.3, found 498.3 |
| 158 | 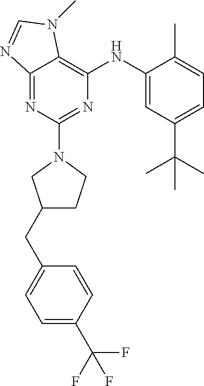 | N-(5-tert-butyl-2- methylphenyl)-7-methyl-2-{3- [4-(trifluoromethyl)benzyl] pyrrolidin-1-yl}-7H-purin-6- amine | Calc'd 523.3, found 523.3 |
| 159 | 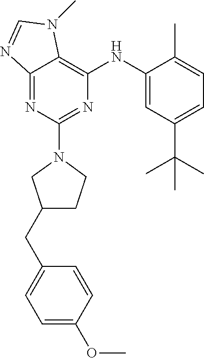 | N-(5-tert-butyl-2- methylphenyl)-2-[3-(4- methoxybenzyl)pyrrolidin-1- yl]-7-methyl-7H-purin-6-amine | Calc'd 485.3, found 485.3 |
| 160 | 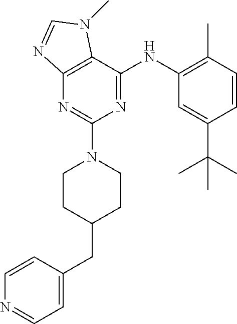 | N-(5-tert-butyl-2- methylphenyl)-7-methyl-2-[4- (pyridin-4-ylmethyl)piperidin- 1-yl]-7H-purin-6-amine | Calc'd 470.3, found 470.3 |
| 161 | 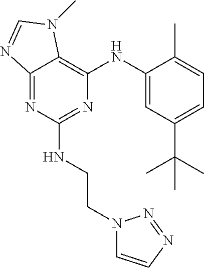 | N~6~-(5-tert-butyl-2- methylphenyl)-7-methyl- N~2~-[2-(1H-1,2,3-triazol-1- yl)ethyl]-7H-purine-2,6- diamine | Calc'd 406.2, found 406.2 |
| 162 | 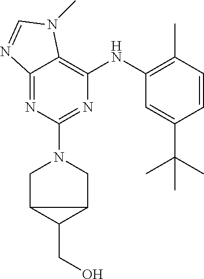 | (3-{6-[(5-tert-butyl-2- methylphenyl)amino]-7- methyl-7H-purin-2-yl}-3- azabicyclo[3.1.0]hex-6- yl)methanol | Calc'd 407.3, found 407.3 |
| 163 | 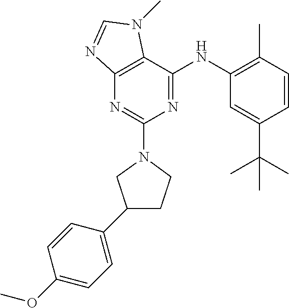 | N-(5-tert-butyl-2- methylphenyl)-2-[3-(4- methoxyphenyl)pyrrolidin-1- yl]-7-methyl-7H-purin-6-amine | Calc'd 471.3, found 471.2 |
| 164 | 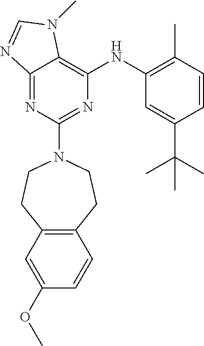 | N-(5-tert-butyl-2- methylphenyl)-2-(7-methoxy- 1,2,4,5-tetrahydro-3H-3- benzazepin-3-yl)-7-methyl-7H- purin-6-amine | Calc'd 471.3, found 471.3 |
| 165 | 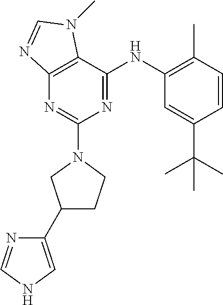 | N-(5-tert-butyl-2- methylphenyl)-2-[3-(1H- imidazol-4-yl)pyrrolidin-1-yl]- 7-methyl-7H-purin-6-amine | Calc'd 431.3, found 431.2 |
| 166 | 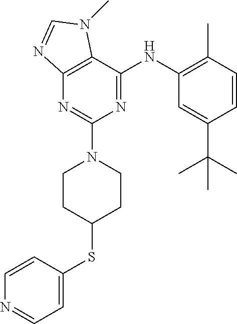 | N-(5-tert-butyl-2- methylphenyl)-7-methyl-2-[4- (pyridin-4-ylthio)piperidin-1- yl]-7H-purin-6-amine | Calc'd 488.3, found 488.2 |
| 167 | 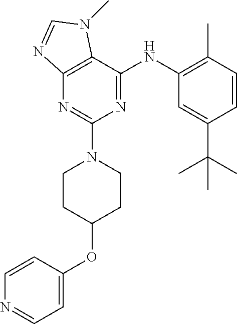 | N-(5-tert-butyl-2- methylphenyl)-7-methyl-2-[4- (pyridin-4-yloxy)piperidin-1- yl]-7H-purin-6-amine | Calc'd 472.3, found 472.2 |
| 168 | 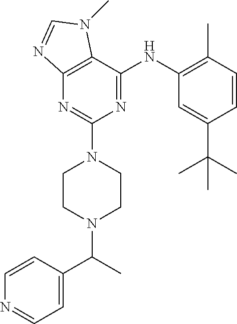 | N-(5-tert-butyl-2- methylphenyl)-7-methyl-2-[4- (1-pyridin-4-ylethyl)piperazin- 1-yl]-7H-purin-6-amine | Calc'd 485.3, found 485.3 |
| 169 | 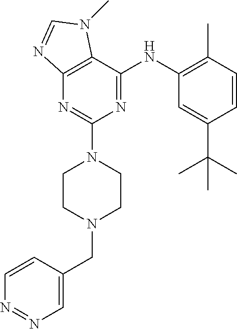 | N-(5-tert-butyl-2- methylphenyl)-7-methyl-2-[4- (pyridazin-4- ylmethyl)piperazin-1-yl]-7H- purin-6-amine | Calc'd 472.3, found 472.3 |
| 170 | 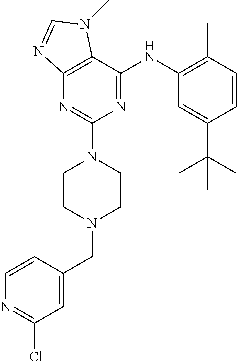 | N-(5-tert-butyl-2- methylphenyl)-2-{4-[(2- chloropyridin-4- yl)methyl]piperazin-1-yl}-7- methyl-7H-purin-6-amine | Calc'd 505.3, found 505.2 |
| 171 | 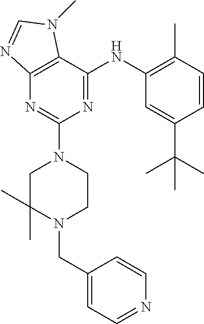 | N-(5-tert-butyl-2- methylphenyl)-2-[3,3- dimethyl-4-(pyridin-4- ylmethyl)piperazin-1-yl]-7- methyl-7H-purin-6-amine | Calc'd 499.3, found 499.3 |
| 172 | 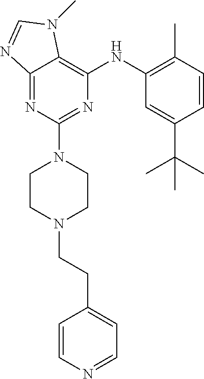 | N-(5-tert-butyl-2- methylphenyl)-7-methyl-2-[4- (2-pyridin-4-ylethyl)piperazin- 1-yl]-7H-purin-6-amine | Calc'd 485.3, found 485.3 |
| 173 | 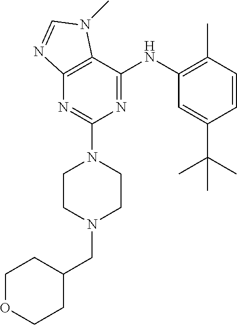 | N-(5-tert-butyl-2- methylphenyl)-7-methyl-2-[4- (tetrahydro-2H-pyran-4- ylmethyl)piperazin-1-yl]-7H- purin-6-amine | Calc'd 478.3, found 478.3 |
| 174 | 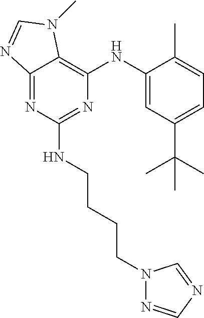 | N~6~-(5-tert-butyl-2- methylphenyl)-7-methyl- N~2~-[4-(1H-1,2,4-triazol-1- yl)butyl]-7H-purine-2,6- diamine | Calc'd 434.3, found 434.2 |
| 175 | 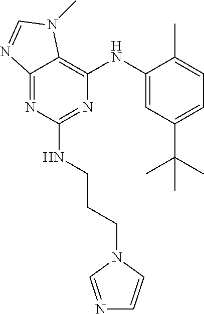 | N~6~-(5-tert-butyl-2- methylphenyl)-N~2~-[3-(1H- imidazol-1-yl)propyl]-7- methyl-7H-purine-2,6-diamine | Calc'd 419.3, found 419.2 |
| 176 | 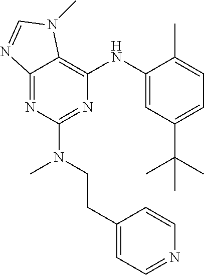 | N~6~-(5-tert-butyl-2- methylphenyl)-N~2~,7- dimethyl-N~2~-(2-pyridin-4- ylethyl)-7H-purine-2,6- diamine | Calc'd 430.3, found 430.2 |
| 177 | 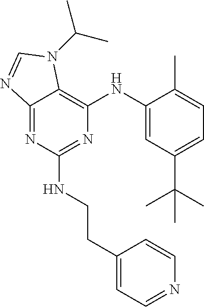 | N~6~-(5-tert-butyl-2- methylphenyl)-7-isopropyl- N~2~-(2-pyridin-4-ylethyl)- 7H-purine-2,6-diamine | Calc'd 444.3, found 444.2 |
| 178 | 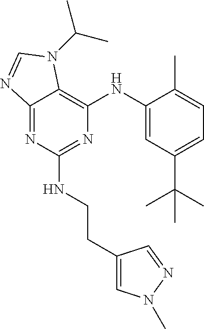 | N~6~-(5-tert-butyl-2- methylphenyl)-7-isopropyl- N~2~-[2-(1-methyl-1H- pyrazol-4-yl)ethyl]-7H-purine- 2,6-diamine | Calc'd 447.3, found 447.3 |
| 179 | 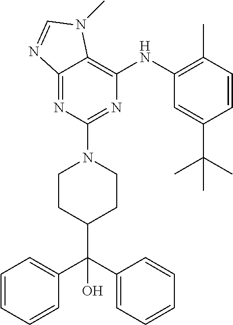 | (1-{6-[(5-tert-butyl-2- methylphenyl)amino]-7- methyl-7H-purin-2- yl}piperidin-4- yl)(diphenyl)methanol | Calc'd 561.3, found 561.3 |
| 180 | 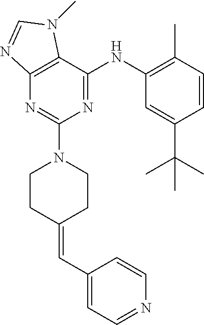 | N-(5-tert-butyl-2- methylphenyl)-7-methyl-2-[4- (pyridin-4-ylmethylene) piperidin-1-yl]-7H-purin-6-amine | Calc'd 468.3, found 468.2 |
| 181 | 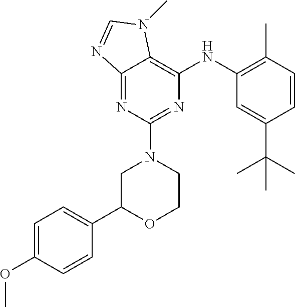 | N-(5-tert-butyl-2- methylphenyl)-2-[2-(4- methoxyphenyl)morpholin-4- yl]-7-methyl-7H-purin-6-amine | Calc'd 487.3, found 487.0 |
| 182 | 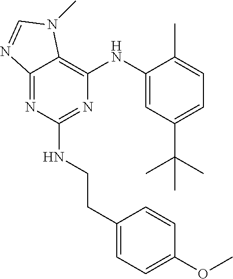 | N~6~-(5-tert-butyl-2- methylphenyl)-N~2~-[2-(4- methoxyphenyl)ethyl]-7- methyl-7H-purine-2,6-diamine | Calc'd 445.3, found 445.2 |
| 183 | 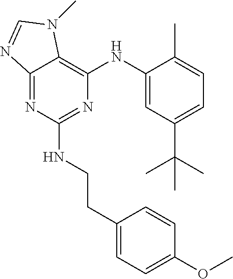 | N~6~-(5-tert-butyl-2- methylphenyl)-N~2~-[2-(4- methoxyphenyl)ethyl]-7- methyl-7H-purine-2,6-diamine | Calc'd 445.3, found 445.3 |
| 184 | 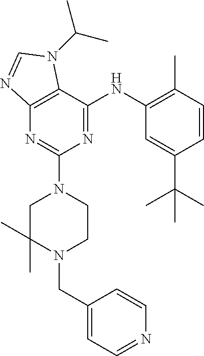 | N-(5-tert-butyl-2- methylphenyl)-2-[3,3- dimethyl-4-(pyridin-4- ylmethyl)piperazin-1-yl]-7- isopropyl-7H-purin-6-amine | Calc'd 527.4, found 527.3 |
| 185 | 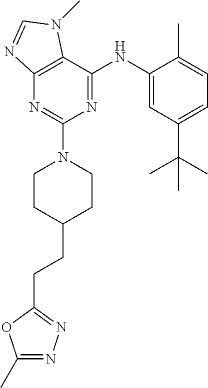 | N-(5-tert-butyl-2- methylphenyl)-7-methyl-2-{4- [2-(5-methyl-1,3,4-oxadiazol- 2-yl)ethyl]piperidin-1-yl}-7H- purin-6-amine | Calc'd 489.3, found 489.3 |
| 186 | 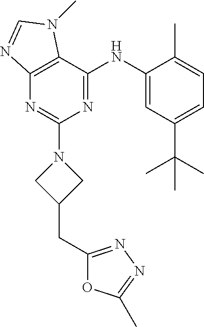 | N-(5-tert-butyl-2- methylphenyl)-7-methyl-2-{3- [(5-methyl-1,3,4-oxadiazol-2- yl)methyl]azetidin-1-yl}-7H- purin-6-amine | Calc'd 447.3, found 447.2 |
| 187 | 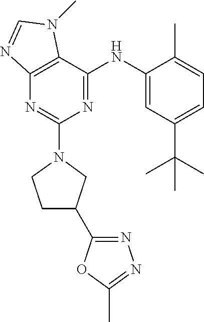 | N-(5-tert-butyl-2- methylphenyl)-7-methyl-2-[3- (5-methyl-1,3,4-oxadiazol-2- yl)pyrrolidin-1-yl]-7H-purin-6- amine | Calc'd 447.3, found 447.2 |
| 188 | 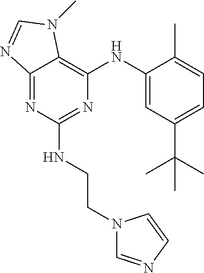 | N~6~-(5-tert-butyl-2- methyphenyl)-N~2~-[2-(1H- imidazol-1-yl)ethyl]-7-methyl- 7H-purine-2,6-diamine | Calc'd 405.3, found 405.2 |
| 189 | 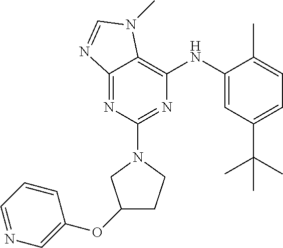 | N-(5-tert-butyl-2- methylphenyl)-7-methyl-2-[3- (pyridin-3-yloxy)pyrrolidin-1- yl]-7H-purin-6-amine | Calc'd 458.3, found 458.3 |
| 190 | 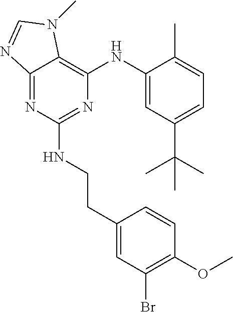 | N~2~-[2-(3-bromo-4- methoxyphenyl)ethyl]-N~6~- (5-tert-butyl-2-methylphenyl)- 7-methyl-7H-purine-2,6- diamine | Calc'd 523.2, found 523.2 |
| 191 | 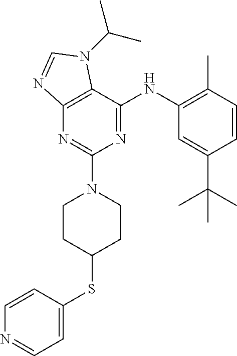 | N-(5-tert-butyl-2- methylphenyl)-7-isopropyl-2- [4-(pyridin-4-ylthio)piperidin- 1-yl]-7H-purin-6-amine | Calc'd 516.3, found 516.2 |
| 192 | 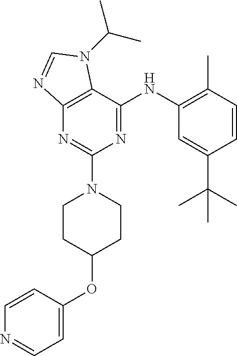 | N-(5-tert-butyl-2- methylphenyl)-7-isopropyl-2- [4-(pyridin-4-yloxy)piperidin- 1-yl]-7H-purin-6-amine | Calc'd 500.3, found 500.2 |
| 193 | 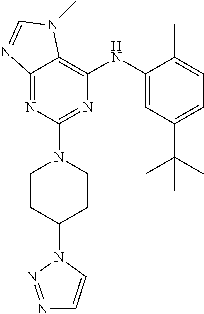 | N-(5-tert-butyl-2- methylphenyl)-7-methyl-2-[4- (1H-1,2,3-triazol-1- yl)piperidin-1-yl]-7H-purin-6- amine | Calc'd 446.3, found 446.2 |
| 194 | 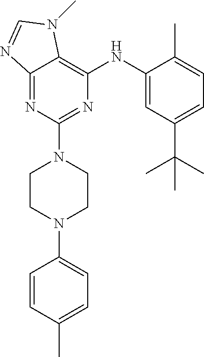 | N-(5-tert-butyl-2- methylphenyl)-7-methyl-2-[4- (4-methylphenyl)piperazin-1- yl]-7H-purin-6-amine | Calc'd 470.3, found 470.3 |
| 195 | 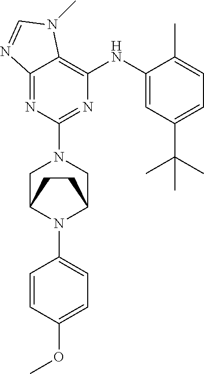 | N-(5-tert-butyl-2- methylphenyl)-2-[(1R,5S)-8- (4-methoxyphenyl)-3,8- diazabicyclo[3.2.1]oct-3-yl]-7- methyl-7H-purin-6-amine | Calc'd 512.3, found 512.3 |
| 196 | 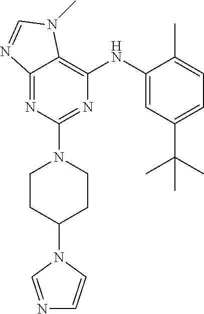 | N-(5-tert-butyl-2- methylphenyl)-2-[4-(1H- imidazol-1-yl)piperidin-1-yl]- 7-methyl-7H-purin-6-amine | Calc'd 445.3, found 445.3 |
| 197 | 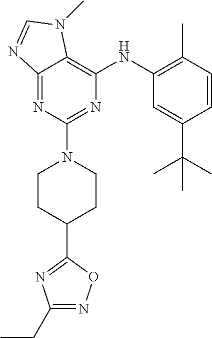 | N-(5-tert-butyl-2- methylphenyl)-2-[4-(3-ethyl- 1,2,4-oxadiazol-5-yl)piperidin- 1-yl]-7-methyl-7H-purin-6- amine | Calc'd 475.3, found 475.1 |
| 198 | 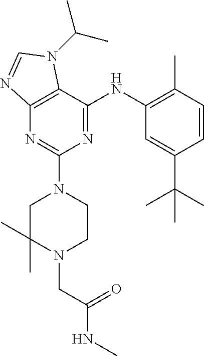 | 2-(4-{6-[(5-tert-butyl-2- methylphenyl)amino]-7- isopropyl-7H-purin-2-yl}-2,2- dimethylpiperazin-1-yl)-N- methylacetamide | Calc'd 507.4, found 507.3 |
| 199 | 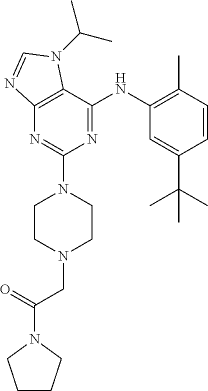 | N-(5-tert-butyl-2- methylphenyl)-7-isopropyl-2- [4-(2-oxo-2-pyrrolidin-1- ylethyl)piperazin-1-yl]-7H- purin-6-amine | Calc'd 519.4, found 519.3 |
| 200 | 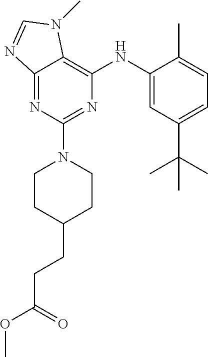 | methyl 3-(1-{6-[(5-tert-butyl- 2-methylphenyl)amino]-7- methyl-7H-purin-2- yl}piperidin-4-yl)propanoate | Calc'd 465.3, found 465.2 |
| 201 | 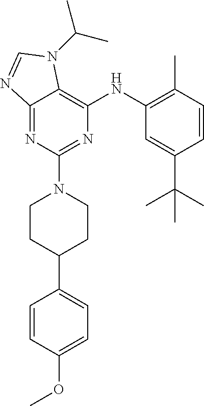 | N-(5-tert-butyl-2- methylphenyl)-7-isopropyl-2- [4-(4-methoxyphenyl)piperidin- 1-yl]-7H-purin-6-amine | Calc'd 513.3, found 513.2 |
| 202 | 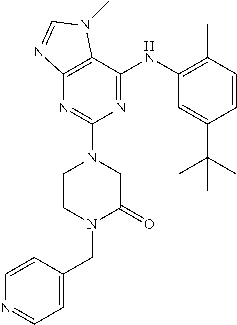 | 4-{6-[(5-tert-butyl-2- methylphenyl)amino]-7- methyl-7H-purin-2-yl}-1- (pyridin-4-ylmethyl)piperazin- 2-one | Calc'd 485.3, found 485.2 |
| 203 | 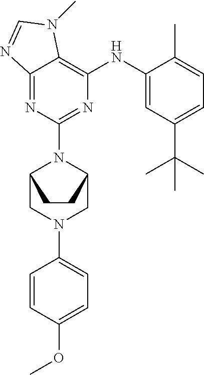 | N-(5-tert-butyl-2- methylphenyl)-2-[(1R,5S)-3- (4-methoxyphenyl)-3,8- diazabicyclo[3.2.1]oct-8-yl]-7- methyl-7H-purin-6-amine | Calc'd 512.3, found 512.3 |
| 204 | 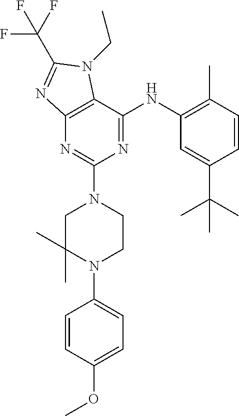 | N-(5-tert-butyl-2- methylphenyl)-7-ethyl-2-[4-(4- methoxyphenyl)-3,3- dimethylpiperazin-1-yl]-8- (trifluoromethyl)-7H-purin-6- amine | Calc'd 596.3, found 596.3 |
| 205 | 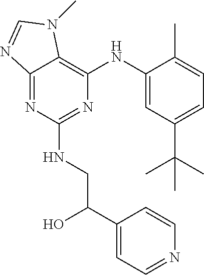 | 2-({6-[(5-tert-butyl-2- methylphenyl)amino]-7- methyl-7H-purin-2-yl}amino)- 1-pyridin-4-ylethanol | Calc'd 432.3, found 432.2 |
| 206 | 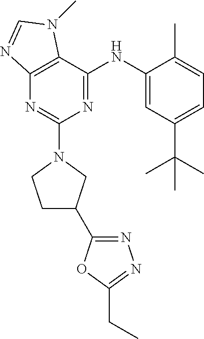 | N-(5-tert-butyl-2- methylphenyl)-2-[3-(5-ethyl- 1,3,4-oxadiazol-2- yl)pyrrolidin-1-yl]-7-methyl- 7H-purin-6-amine | Calc'd 461.3, found 461.2 |
| 207 | 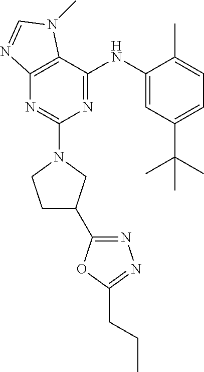 | N-(5-tert-butyl-2- methylphenyl)-7-methyl-2-[3- (5-propyl-1,3,4-oxadiazol-2- yl)pyrrolidin-1-yl]-7H-purin-6- amine | Calc'd 475.3, found 475.3 |
| 208 | 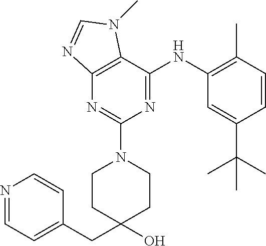 | 1-{6-[(5-tert-butyl-2- methylphenyl)amino]-7- methyl-7H-purin-2-yl}-4- (pyridin-4-ylmethyl)piperidin- 4-ol | Calc'd 486.3, found 486.3 |
| 209 | 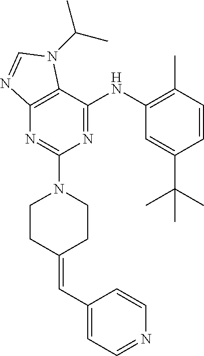 | N-(5-tert-butyl-2- methylphenyl)-7-isopropyl-2- [4-(pyridin-4- ylmethylene)piperidin-1-yl]- 7H-purin-6-amine | Calc'd 496.3, found 496.3 |
| 210 | 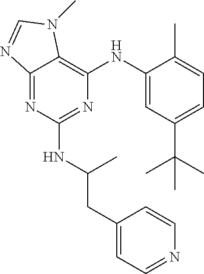 | N~6~-(5-tert-butyl-2- methylphenyl)-7-methyl- N~2~-(1-methyl-2-pyridin-4- ylethyl)-7H-purine-2,6- diamine | Calc'd 430.3, found 430.2 |
| 211 | 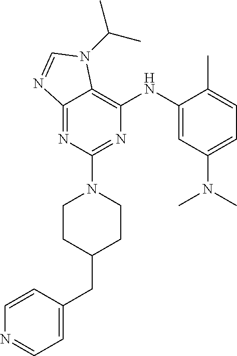 | N~3~-{7-isopropyl-2-[4- (pyridin-4-ylmethyl)piperidin- 1-yl]-7H-purin-6-yl}- N~1~,N~1~,4- trimethylbenzene1,3-diamine | Calc'd 485.3, found 485.3 |
| 212 | 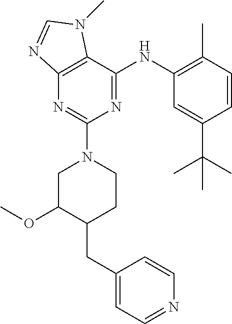 | N-(5-tert-butyl-2- methylphenyl)-2-[3-methoxy- 4-(pyridin-4- ylmethyl)piperidin-1-yl]-7- methyl-7H-purin-6-amine | Calc'd 500.3, found 500.3 |
| 213 | 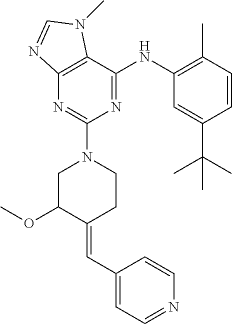 | N-(5-tert-butyl-2- methylphenyl)-2-[(4E)-3- methoxy-4-(pyridin-4- ylmethylene)piperidin-1-yl]-7- methyl-7H-purin-6-amine | Calc'd 498.3, found 498.2 |
| 214 | 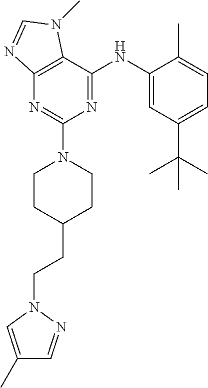 | N-(5-tert-butyl-2- methylphenyl)-7-methyl-2-{4- [2-(4-methyl-1H-pyrazol-1- yl)ethyl]piperidin-1-yl}-7H- purin-6-amine | Calc'd 487.3, found 487.3 |
| 215 | 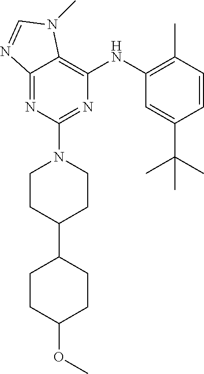 | N-(5-tert-butyl-2- methylphenyl)-2-[4-(4- methoxycyclohexyl)piperidin- 1-yl]-7-methyl-7H-purin-6- amine | Calc'd 491.3, found 491.3 |
| 216 | 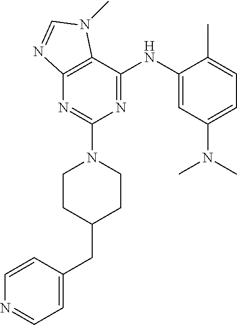 | N~1~,N~1~,4-trimethyl-N~3~- {7-methyl-2-[4-(pyridin-4- ylmethyl)piperidin-1-yl]-7H- purin-6-yl}benzene-1,3- diamine | Calc'd 457.3, found 457.2 |
| 217 | 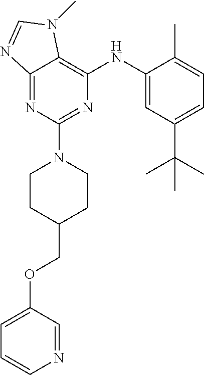 | N-(5-tert-butyl-2- methylphenyl)-7-methyl-2-{4- [(pyridin-3- yloxy)methyl]piperidin-1-yl}- 7H-purin-6-amine | Calc'd 486.3, found 486.2 |
| 218 | 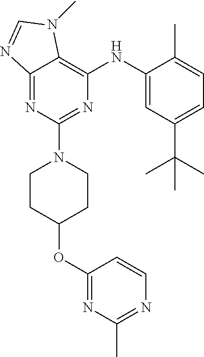 | N-(5-tert-butyl-2- methylphenyl)-7-methyl-2-{4- [(2-methylpyrimidin-4- yl)oxy]piperidin-1-yl}-7H- purin-6-amine | Calc'd 487.3, found 487.2 |
| 219 | 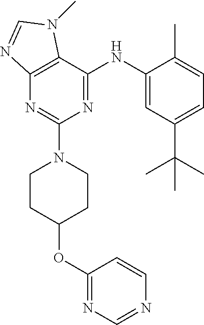 | N-(5-tert-butyl-2- methylphenyl)-7-methyl-2-[4- (pyrimidin-4-yloxy)piperidin- 1-yl]-7H-purin-6-amine | Calc'd 473.3, found 473.2 |
| 220 | 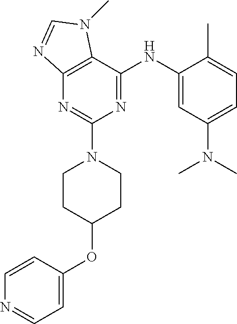 | N~1~,N~1~,4-trimethyl-N~3~- {7-methyl-2-[4-(pyridin-4- yloxy)piperidin-1-yl]-7H- purin-6-yl}benzene-1,3- diamine | Calc'd 459.3, found 459.2 |
| 221 | 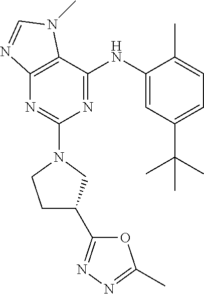 | N-(5-tert-butyl-2- methylphenyl)-7-methyl-2- [(3R)-3-(5-methyl-1,3,4- oxadiazol-2-yl)pyrrolidin-1- yl]-7H-purin-6-amine | Calc'd 447.3, found 447.2 |
| 222 | 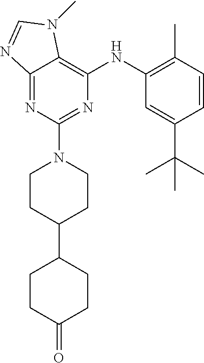 | 4-(1-{6-[(5-tert-butyl-2- methylphenyl)amino]-7- methyl-7H-purin-2- yl}piperidin-4- yl)cyclohexanone | Calc'd 475.3, found 475.3 |
| 223 | 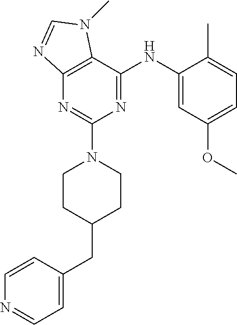 | N-(5-methoxy-2- methylphenyl)-7-methyl-2-[4- (pyridin-4-ylmethyl)piperidin- 1-yl]-7H-purin-6-amine | Calc'd 444.3, found 444.2 |
| 224 | 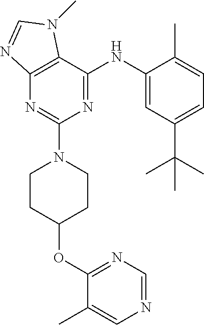 | N-(5-tert-butyl-2- methylphenyl)-7-methyl-2-{4- [(5-methylpyrimidin-4- yl)oxy]piperidin-1-yl}-7H- purin-6-amine | Calc'd 487.3, found 487.3 |
| 225 | 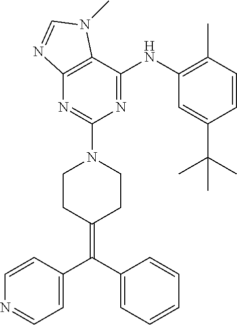 | N-(5-tert-butyl-2- methylphenyl)-7-methyl-2-{4- [phenyl(pyridin-4- yl)methylene]piperidin-1-yl}- 7H-purin-6-amine | Calc'd 544.3, found 544.3 |
| 226 | 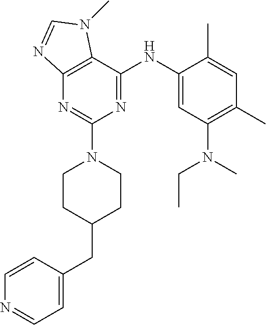 | N-ethyl-N,4,6-trimethyl-N′-{7- methyl-2-[4-(pyridin-4- ylmethyl)piperidin-1-yl]-7H- purin-6-yl}benzene-1,3- diamine | Calc'd 485.3, found 485.3 |
| 227 | 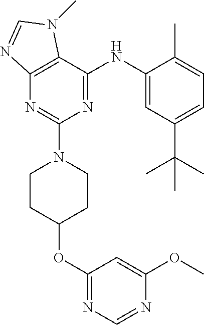 | N-(5-tert-butyl-2- methylphenyl)-2-{4-[(6- methoxypyrimidin-4- yl)oxy]piperidin-1-yl}-7- methyl-7H-purin-6-amine | Calc'd 503.3, found 503.3 |
| 228 | 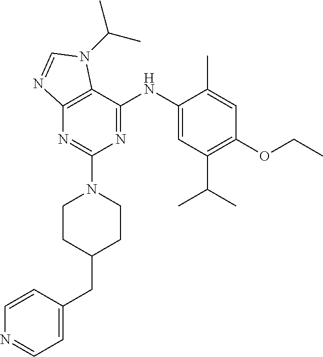 | N-(4-ethoxy-5-isopropyl-2- methylphenyl)-7-isopropyl-2- [4-(pyridin-4- ylmethyl)piperidin-1-yl]-7H- purin-6-amine | Calc'd 528.3, found 528.4 |
| 229 | 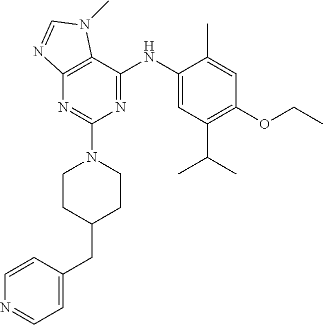 | N-(4-ethoxy-5-isopropyl-2- methylphenyl)-7-methyl-2-[4- (pyridin-4-ylmethyl)piperidin- 1-yl]-7H-purin-6-amine | Calc'd 500.3, found 500.3 |
| 230 | 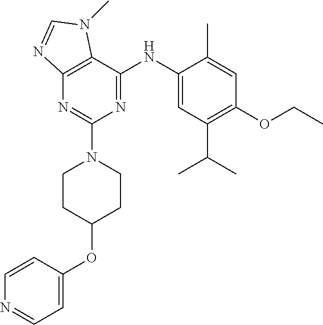 | N-(4-ethoxy-5-isopropyl-2- methylphenyl)-7-methyl-2-[4- (pyridin-4-yloxy)piperidin-1- yl]-7H-purin-6-amine | Calc'd 502.3, found 502.3 |
| 231 | 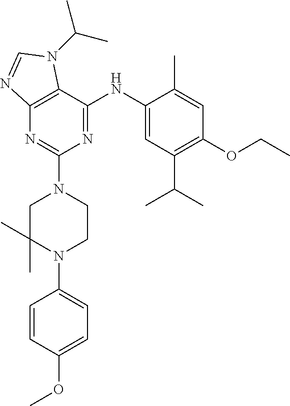 | N-(4-ethoxy-5-isopropyl-2- methylphenyl)-7-isopropyl-2- [4-(4-methoxyphenyl)-3,3- dimethylpiperazin-1-yl]-7H- purin-6-amine | Calc'd 572.4, found 572.5 |

### Example 232

### N-(5-tert-butyl-2-methylphenyl)-7-methyl-2-[4-(1,3,4-oxadiazol-2-ylmethyl)piperidin-1-yl]-7H-purin-6-amine

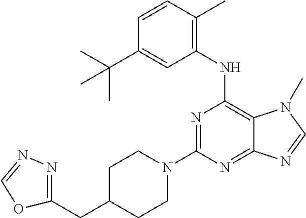

### Step 1

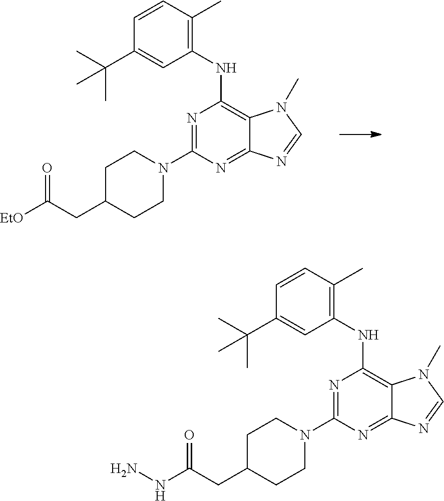

To a solution of the ethyl ester (Example 59) (100 mg, 0.22 mmol) in MeOH (5 mL) was added hydrazine hydrate (215 mg, 4.30 mmol). The reaction mixture was refluxed for 72 h and then concentrated to give 90 mg (93%) of the desired hydrazide as light brown crystals confirmed by MS (ESI+): card [M+H]⁺ 451.3, obs. 451.2.

### Step 2

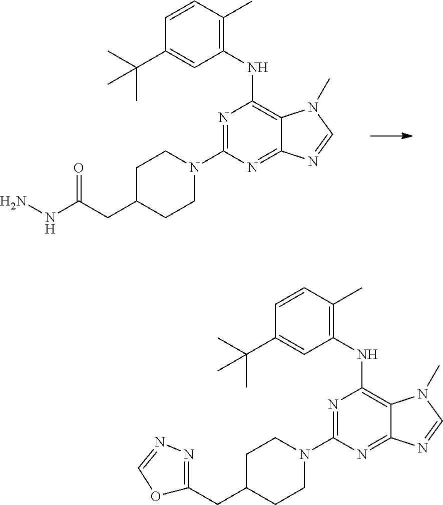

A solution of the hydrazide (90 mg, 0.20 mmol) in trimethyl orthoformate (10.0 mL, 90.0 mmol) was refluxed for 24 h and concentrated to give 90 mg (98%) of the desired oxadiazole as light brown crystals confirmed by MS (ESI+): [M+H]⁺ 461.3, obs. 461.2 and ¹H NMR (DMSO-d₆) [symbol] 7.92 (s, 1H), 7.80 (s, 1H), 7.59 (d, 1H, J=1.6), 7.16-7.04 (1H, m), 7.07 (dd, 1H, J₁=8.0 Hz, J₂=1.8 Hz), 4.43 (d, 2H, J=12.9 Hz), 3.97 (s, 3H), 2.78 (d, 2H, J=7.0 Hz), 2.64 (t, 2H, J=11.7 Hz), 2.17 (s, 3H), 1.96-1.86 (m, 1H), 1.55 (d, 2H, J=11.3), 1.23 (s, 9H), 1.14-1.02 (m, 1H).

1. A compound of formula IA or IB: 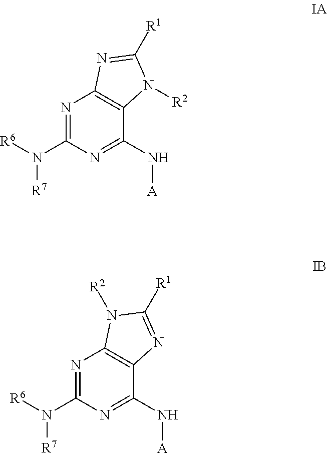
or a pharmaceutically acceptable salt or hydrate thereof; wherein:
R¹ represents H, CF₃ or C₁₋₄alkyl;
R² represents H, C₁₋₆alkyl or C₃₋₆cycloalkyl, either of which optionally bears a substituent selected from halogen, CF₃, C₁₋₄alkoxy and C₁₋₄alkoxycarbonyl;
A represents a group selected from: 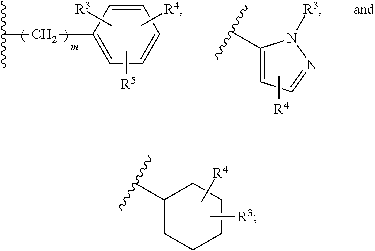
m is 0 or 1;
R³ represents C₁₋₆alkyl;
R⁴ and R⁵ are independently selected from H, halogen, C₁₋₆alkyl, C₃₋₆cycloalkyl, C₁₋₆alkoxy, C₁₋₆alkylamino and di(C₁₋₆alkyl)amino;
R⁶ represents H or C₁₋₆alkyl;
R⁷ represents —(CO)ₙ-L1-X;
n is 0 or 1;
L1 represents a divalent linking group selected from cyclopropane-1,2-diyl and C₁₋₆alkylene which optionally bears up to 2 substituents independently selected from OH, ═O, F and C₁₋₄alkyl;
X represents C₁₋₄alkoxy, C₃₋₆cycloalkylC₁₋₄alkoxy, tetrahydrofuryl, tetrahydropyranyl, Ar, ArO or ArNH;
or R⁶ and R⁷ together with the nitrogen atom which they are mutually attached complete a ring represented by: 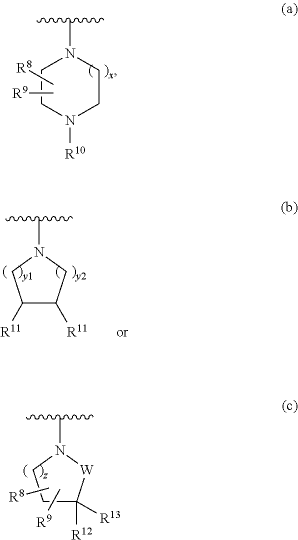
x is 1 or 2;
y1 and y2 are independently 1 or 2;
z is 0, 1 or 2;
W represents CH₂, CH₂CH₂ or CH₂CH₂O with the proviso that z=0 when W represents CH₂CH₂O;
R⁸ and R⁹ are attached to the same carbon atom or to different carbon atoms and independently represent H, halogen, CF₃, C₁₋₄alkyl or C₁₋₄alkoxy; or when attached to the same carbon atom R⁸ and R⁹ may together represent ═O; or when attached to different carbon atoms R⁸ and R⁹ may together represent a —CH₂CH₂— or —CH₂CH₂CH₂— bridge;
R¹⁰ represents a group -L2-Y;
Y represents H, Ar, OAr, NHAr, SAr, SO₂Ar, OR^{a}, N(R^{a})₂, CN, halogen, CF₃, COR^{a}, CO₂R^{a}, SO₂R^{a}, diphenylhydroxymethyl, C₃₋₆cycloalkyl, tetrahydrofuryl or tetrahydropyranyl, said C₃₋₆cycloalkyl, tetrahydrofuryl or tetrahydropyranyl optionally bearing up to 3 substituents independently selected from halogen, CF₃, C₁₋₄alkyl, oxo and C₁₋₄alkoxy;
L2 represents a bond or C₁₋₆alkylene which optionally bears up to 3 substituents selected from halogen, C₁₋₄alkyl, OH and ═O, with the proviso that L2 cannot represent a bond unless Y represents H, Ar, COR^{a}, CO₂R^{a}, SO₂R^{a} or C₃₋₆cycloalkyl;
the two R¹¹ groups together represent a fused carbocyclic or heterocyclic ring of up to 6 ring atoms in total which optionally bears up to 2 substituents independently selected from halogen, CF₃, C₁₋₄alkoxy and hydroxyC₁₋₄alkyl;
R¹² represents H or a group —(Z)ₚ-L3-Y;
R¹³ represents, H, OH, Ar or C₁₋₆alkyl;
or R¹² and R¹³ together represent ═CH—Ar or ═C(Ar)₂ where the Ar groups are the same or different;
or R¹² and R¹³ together complete a spiro-linked 5-membered ring in which at least 1 of the ring atoms is N, O or S, said ring optionally being benzo-fused and said ring optionally bearing up to 2 substituents selected from oxo, Ar, CF₃, halogen, C₁₋₄alkyl, C₁₋₄alkoxy and C₁₋₄alkylcarbonyl;
Z represents O, S, SO₂, or NH;
p is 0 or 1;
L3 represents a bond or C₁₋₆alkylene which optionally bears up to 3 substituents selected from halogen, C₁₋₄alkyl, OH and ═O, with the proviso that p is 0 when L3 represents a bond;
Ar represents phenyl or 5- or 6-membered heteroaryl, any of which optionally bears up to 3 substituents selected from halogen, CN, phenyl, R^{b}, OR^{a}, N(R^{a})₂, CO₂R^{a}, CON(R^{a})₂ and SO₂R^{b};
each R^{a} independently represents H or C₁₋₄alkyl, C₃₋₆cycloalkyl, or C₃₋₆cycloalkylC₁₋₄alkyl, any of which optionally bears up to 3 fluorine substituents, or two R^{a} groups attached to the same nitrogen optionally together complete a heterocyclic ring of 4, 5 or 6 members which optionally bears up to 3 substituents independently selected from halogen, C₁₋₄alkyl, C₁₋₄alkoxy, CF₃; and oxo;
and R^{b} represents R^{a} that is other than H; or two R^{b} groups attached to adjacent ring positions may complete a fused 5- or 6-membered ring optionally bearing up to 3 substituents independently selected from halogen, CF₃, C₁₋₄alkyl, oxo and C₁₋₄alkoxy.

2. A compound according to claim 1 wherein R⁶ and R⁷ complete a ring selected from:

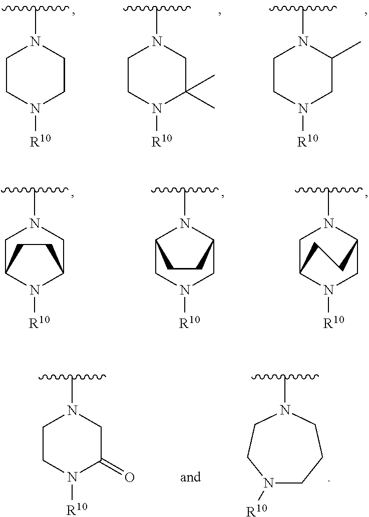

3. A compound according to claim 1 wherein R⁶ and R⁷ complete a ring selected from:

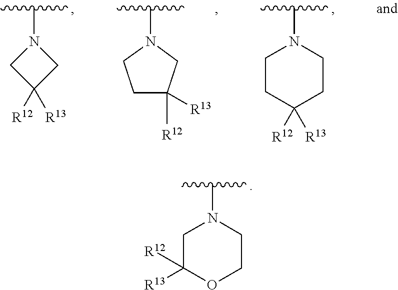

4. A compound according to claim 2 wherein R¹⁰ represents Ar or ArCH₂.

5. A compound according to claim 4 wherein R¹⁰ represents 4-methoxyphenyl or 4-pyridylmethyl.

6. A compound according to claim 3 wherein R¹² represents Ar or ArS and R¹³ is H.

7. A compound according to claim 6 wherein R¹² represents 4-pyridyl or 4-pyridylthio.

8. A compound according to claim 1 wherein R⁶ is H and R⁷ represents —(CO)ₙ-L1-X.

9. A compound according to claim 8 wherein X represents Ar.

10. A compound according to claim 9 wherein R⁷ represents 2-(4-pyridyl)ethyl or 2-(1-methyl-1H-pyrazol-4-yl)ethyl.

11. A compound according to claim 1 wherein A represents

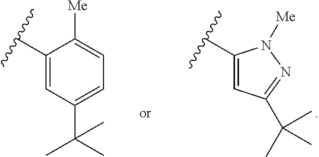

12. A pharmaceutical composition comprising a compound according to claim 1 and a pharmaceutically-acceptable carrier.

13. A method of treating or preventing a disease associated with deposition of Aβ in the brain comprising administration to a patient in need thereof a therapeutically effective amount of a compound of formula IA or IB as defined in claim 1 or a pharmaceutically-acceptable salt or hydrate thereof.
